SEQUENCE LISTING (3 sequences)

SEQ ID NO: 1 - DNA, 10655 bp, Hepatitis C virus
acctgctctctatgagagcaacactccaccatgaaccgctcccctgtgaggaactactgtcttcacgcagaaagcgtctagccatggcgttagtatgagtgttgtacagcctccaggaccccccctcccgggagagccatagtggtctgcggaaccggtgagtacaccggaatcgccgggatgaccgggtcctttcttggattaacccgctcaatgcccggaaatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacggcgcgccaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcccctatcactgcgtatgcgcaacagacccgggggacgctaggcaccataatcacaagcctcaccggcagagataccaacgagaactgcggtgaaatccaggtgctgtccacggcgacgcagtctttcttgggcagtgcgatcaatggcgtcatgtggacggtttaccatggggcgggcagcaagaccatcagcggcccgaaaggaccggtcaaccagatgtacaccaatgtcgaccaagacttggtgggctggcccgcacctccaggagtgaagtccttggccccatgcacctgtggctcgtcggacctgttcctggtcaccaggcacgccgacgtggtgcccgtgcgcagaagaggcgacactcgtggcgccctcttaagccctaggccgatttcaactcttaagggatcatccggtgggccactgttgtgccccctgggtcacgtcgccggcatcttccgagccgcggtgtgtacccggggcgtggccaaagcagtggacttcgtaccggttgaatctcttgaaaccaccatgaggtctccagtattctctgacaattccactcctcctgccgtgccccagacttaccaagtagcccacttgcacgcgccaacgggaagtggcaaaagcacaaaagtccctgccgcgtatgcggctcaaggctacaaagtgctagtgctaaacccctctgttgctgcgactctgggttttggggcatatatgtccaaggcacatggcattgatcccaatatacgatcaggggtcagaactatcaccacaggcgcgcccatcacgtactccacgtacgggaagttcttggccgatggaggttgcgcggggggcgcgtatgatatcatcatctgtgatgaatgccattctactgatgcaacgacggtcctgggcataggcacggtcttagaccaagcggaaaccgctggagcgcgtcttgtcgtgctcgcgaccgctacgccacccggatcggtgacaacgccccactccaacatagaggaggtcgctttgccgacgacgggagagatacctttctacggcaaggcagtccccctatctttggttaaggggggcaggcatctcatcttctgtcactcaaagaagaagtgtgatgagttggccaagcaactatcatctcttggcctcaatgcggtagcctattataggggccttgacgtctcagtgataccattatctggagacgtcgtggtttgcgccacagacgccctcatgacaggcttcacaggtgactttgactcagtgatagactgcaatacgtctgtcatacaaacagttgacttcagcctagaccctactttcaccatagagaccacaaccgtaccccaggacgcagtgtcccggagccaacggaggggccgcactggtagggggaggttaggcatataccggtatgtcaccccaggagagaggccatccggcatatttgacacctcagtactctgcgagtgctacgatgctggatgcgcttggtatgaactgacaccggcagagacaacgatcaggttaagggcttatttcaacacaccgggcctccccgtctgccaggatcacctggaattttgggagagcgtctttacgggtctcacccatatagacggtcatttcctatcccagaccaaacaggcgggtgacaactacccttacctggtcgcctaccaggcaacagtctgtgccaaggctttggcacccccacccagttgggacacaatgtggaaatgcctcctccgcctcaagccaactttgcggggaccgacccccctcctttacaggctggggtccgtacaaaatgaggtggtactcacgcacccgatcaccaagtacatcatggcctgcatgtctgccgatcttgaggttgtgaccagcacgtgggtcctggtaggcggtcttctggcggcccttgctgcctactgcttgtcagtgggcagcgtggtaatcgtcgggagggtcgtcatatcgggccaacctgctgtcatccccgatcgggaggtgctgtaccgacagttcgacgaaatggaagagtgctctaagcacgttccattcgtcgagcatggcctgcagctagcggagcaattcaaacagaaggccataggccttatgagcatcgctggcaagcaggcccaggaagcagcaccagtggtccagtcaaattttgccaaacttgaacagttttgggcgaagcatatgtggaacttcatcagtggtattcaataccttgccgggctgtctaccttgccgggcaacccaactattgcttccctcatggcgttcaccgccgcggtcactagccccctaacgacccaacagactctcctattcaacatcttgggaggttgggtggcctcacagatcgcgacccctacggcctctacggcttttgtcataagcggcattgcgggggctgcggtcgggagtgttggcctggggaagatcctagtggacattcttgctggctacggtgccggtgtggccggcgctgtggtcaccttcaagatcatgagcggcgagacaccatcaacagaagacttggtgaacttgctcccagcaatactatcgccgggagccctggtggtaggggtggtatgtgccgcaattttgcggcgtcacgtgggaccgggtgagggagcagttcagtggatgaaccgcttgatcgcattcgcgtcaaggggcaaccacgtggctcccacacactacgttcccgagtccgacgcagcggctcgcgtgactgtcatactatcatccctgactgtgacctcccttctcagacgcctccacaagtggatcaacgaggactgttctactccttgtgatcgctcttggttatgggagatctgggactgggtctgcaccgtactgagtgactttaaaacgtggctgaaggccaagctattgcctcgcatgcccggcattcccttcctctcctgtcagagggggtacagaggagtgtggcggggagatggggtaatgcacacaacatgcacatgcggcgcagagctggccggccacgtcaaaaatggctcgatgaggatcgtcgggcccaagacctgcagcaatacctggcacgggaccttccccatcaatgcttacaccacgggtcctagcgtgcccatccccgcgcctaactacaagtttgcgctgtggagggtatccgcggaggaatacgtggaggttcgcagagtaggggagttccattatatcaccggggttacacaggataacatcaagtgcccctgccaggtacccgcacctgagttcttcactgaggtggatggcgtcaggctccatcgtcatgcccctgcgtgcaagcccatactgagggacgatgtgtcctttacagtgggcctcaatacttttgtggtggggtcccagctcccctgcgagcccgagccagacgtcgcagtgttaacatctatgctgacagatccatctcacatcacagcggaggcggcacgccgtaggctgggaagggggtcaccaccctccttggccagctcctcggcgagccagctatctgccccatccttaaaagctacatgcaccgaccacaaagactcccctggagtggacctcatcgaggctaatctcctctggggcgccaatgctaccagggttgagtcagaggataaggtgctgatcttggactcttttgagcccctagtggccgagacggatgacagggagatctccgtctcagcagagatcctgcggacttcgaagaagttcccgagggccatgccaatttgggctcagccagcttataacccgcctctcattgagacgtggaaacaaccagactacgaaccaccagtcgttcacggctgcgcactgcccccggacaaaccaactcctgttcctccccccaggaggaagcgggcagttgcgctctcggagtccaacatctcagcggcactggcgagcttggcagacaagacctttagccagccagctgtcagctccgattccggagcggccttttccaccccaactgagacttctgaaccagaccccatcatcgtggacgacaaatcagacgacggatcttactcgtcaatgcctccgcttgaaggggagcctggtgacccagacttgacatcagactcttggtccaccgtcagcggatcggaggacgtagtgtgctgctcaatgtcctactcgtggacgggggcgcttgtcaccccctgcgcagctgaggaaaccaagctgcccatcaaccccctgagcaactcactgctacgccatcacaacatggtgtactccacgacttctcgttccgccgccacccggcagaagaaggtcaccttcgaccgcatgcaagtggtggacagccattacaatgaagtacttaaggagattaaggcacaagcctccacagtgaaggcgcggttactcacggttgaggaagcctgcaacctgacgcccccccactcggccagatcaaaatttggttacggggcgaaggaggttcggagccatacccgcaaagccattaaccacatcaactccgtgtgggaggacttgcgggaagacaacactacccccatccctacaacaatcatggctaagaatgaggtcttctccgtgacaccggagaagggcggcaaaaaatcggctcgtctaatcgtgtaccctgacctaggggtgcgggtgtgcgagaagagggccctgtatgatgccgtcaaacaactttctctggccgtgatgggaacctcttacggtttccagtactcaccatcgcagcgggtcgagttccttttgaacgcttggcgttcaaaaaagacccctatggggttttcatatgacacccgctgctttgactccactgtaaccgaaagggacatcagggttgaggaggaggtctatcagtgttgtgacctagagcccgaagcccgcaaggtgatatccgccctcacggagagactctacgtgggcggtcccatgtacaacagcaggggagacctttgcgggatccgacggtgccgcgcaagcggcgtcttcaccaccagctttgggaacacactaacgtgctatcttaaggccaacgcagccatcagggctgcaggcctaaaagactgcaccatgctggtttgtggcgacgacttagtcgttatcgctgaaagcgatggcgtggaggaggacaaacgtgccctcggagccttcacggaggctatgacgaggtactcagccccccccggagacgccccacaaccagcatatgacctggagctcataacatcttgctcctccaatgtttccgtcgcacatgatgggaccggcaaaagggtctactacctgacccgcaaccctgagactcccctggcacgggctgcctgggagacagctcgacacactccagtcaactcttggcttgggaacatcataatctacgcgcccaccatttgggtgcgcatggttttgatgacccacttcttctcaatactccaaagccaggaggcccttgagaaagcactagacttcgacatgtacggagtcacatactctatcactccgctggacttgccagccataattcaaagactccacggcttaagcgcatttacgctgcacggatactctccacacgaactcaaccgggtggccggaagcctcaggaaacttggggtaccaccgttgagagcgtggagacatcgggcccgagcagtccgcgctaagctcatcgctcaggggggtagagccagaatctgtggcatatacctctttaactgggcggtaaaaaccaaagccaaactcactccattgcccgccgctgccaaactcgacctgtcgagttggtttacggtgggtgctggcgggggggacatttatcacagcgtgtcccatgcccgaccccgctacttactcctgtgcctactcctactttccgtaggggtaggcatcttcctgctgcccgctcggtaggcagcttaacactccgaccttagggtccccttgttttttttttttttttttttttttttttttttttttttttttttttcctttccttctttcctttcctaatctttctttcttggtggctccatcttagccctagtcacggctagctgtgaaaggtccgtgagccgcatgactgcagagagtgctgatactggcctctctgcagatcatgttctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctaatacgactcactata

SEQ ID NO: 2 - protein, 3006 aa, Hepatitis C virus
  MOD_RES (2295)..(2295): Any amino acid
MSTNPKPQRKTKRNTNRRPMDVKFPGGGQIVGGVYLLPRRGPRLGVRATRKTSERSQPRGRRQPIPKARQPEGRSWAQPGYPWPLYGNEGCGWAGWLLSPRGSRPSWGPNDPRRRSRNLGKVIDTLTCGFADLMGYIPVVGAPVGGVARALAHGVRLLEDGVNYATGNLPGCSFSIFLLALLSCLTVPASAYNYRNSSGVYHVTNDCPNSSIVYEADHHILHLPGCVPCVRVGNKSTCWVSLTPTVAAPYLNAPLESLRRHVDLMVGAATLCSALYIGDVCGGAFLVGQLFTFQPRRHWTTQDCNCSIYTGHITGHRMAWDMMMNWSPTTTLVLAQLMRIPSAMVDLLAGGHWGILVGIAYFSMQANWATVILVLFLFAGVDAETIVSGGQAGRTMFGFTSLLNLGPSQKLQLINTNGSWHINRTALNCNDSLNTGLIASLFYAHRFNSSGCPERLASCRSLDSFQQGWGPLGIYQANQSDTRPYCWNYTPQPCWTVPASTVCGPVYCFTPSPVVVGTTDRLGVPTYTWGENETDVFLLNSTRPPRGAWFGCTWMNGTGFTKSCGGPPCRITTINNTWGCPTDCFRKHPEATYIKCGSGPWLTPRCLVHYPYRLWHYPCTVNYTIFKIRMYVGGIEHRLDVACNWTRGEPCDLEHRDRAEISPLLLSTTQWQVLPCSFTTLPALSTGLIHLHQNIVDVQYLYGVGSAVVSWALKWEYIVLAFLLLADARLCACLWMMLMVSQVEAALANLITINAVSVAGIHGFWYAIFVICIAWHVKGRFPAAVTYAACGLWPLLLLVLMLPERAYAFDREIAGSAGGGVLVLLTLLTLSSHYKQWLARGIWWLQYFIARAEAITHVYVPSLDVRGPRDSIIILTALAFPHVAFETTKHLLAILGPLYILQASLLCVPYFVRAHALVKLCSLVRGVMCGKYCQMALLKIGALTGTYVYNHLTPLSDWAAEGLNDLAVALEPVVFTAMEKKIITWGADTAACGDILQGLPVSARLGNEILLGPADAHATRGWRLLAPITAYAQQTRGTLGTIITSLTGRDTNENCGEIQVLSTATQSFLGSAINGVMWTVYHGAGSKTISGPKGPVNQMYTNVDQDLVGWPAPPGVKSLAPCTCGSSDLFLVTRHADVVPVRRRGDTRGALLSPRPISTLKGSSGGPLLCPLGHVAGIFRAAVCTRGVAKAVDFVPVESLETTMRSPVFSDNSTPPAVPQTYQVAHLHAPTGSGKSTKVPAAYAAQGYKVLVLNPSVAATLGFGAYMSKAHGIDPNIRSGVRTITTGAPITYSTYGKFLADGGCAGGAYDIIICDECHSTDATTVLGIGTVLDQAETAGARLVVLATATPPGSVTTPHSNIEEVALPTTGEIPFYGKAVPLSLVKGGRHLIFCHSKKKCDELAKQLSSLGLNAVAYYRGLDVSVIPLSGDVVVCATDALMTGFTGDFDSVIDCNTSVIQTVDFSLDPTFTIETTTVPQDAVSRSQRRGRTGRGRLGIYRYVTPGERPSGIFDTSVLCECYDAGCAWYELTPAETTIRLRAYFNTPGLPVCQDHLEFWESVFTGLTHIDGHFLSQTKQAGDNYPYLVAYQATVCAKALAPPPSWDTMWKCLLRLKPTLRGPTPLLYRLGSVQNEVVLTHPITKYIMACMSADLEVVTSTWVLVGGLLAALAAYCLSVGSVVIVGRVVISGQPAVIPDREVLYRQFDEMEECSKHVPFVEHGLQLAEQFKQKAIGLMSIAGKQAQEAAPVVQSNFAKLEQFWAKHMWNFISGIQYLAGLSTLPGNPTIASLMAFTAAVTSPLTTQQTLLFNILGGWVASQIATPTASTAFVISGIAGAAVGSVGLGKILVDILAGYGAGVAGAVVTFKIMSGETPSTEDLVNLLPAILSPGALVVGVVCAAILRRHVGPGEGAVQWMNRLIAFASRGNHVAPTHYVPESDAAARVTVILSSLTVTSLLRRLHKWINEDCSTPCDRSWLWEIWDWVCTVLSDFKTWLKAKLLPRMPGIPFLSCQRGYRGVWRGDGVMHTTCTCGAELAGHVKNGSMRIVGPKTCSNTWHGTFPINAYTTGPSVPIPAPNYKFALWRVSAEEYVEVRRVGEFHYITGVTQDNIKCPCQVPAPEFFTEVDGVRLHRHAPACKPILRDDVSFTVGLNTFVVGSQLPCEPEPDVAVLTSMLTDPSHITAEAARRRLGRGSPPSLASSSASQLSAPSLKATCTDHKDSPGVDLIEANLLWGANATRVESEDKVLILDSFEPLVAETDDREISVSAEILRTSKKFPRAMPIWAQPAYNPPLIEXWKQPDYEPPVVHGCALPPDKPTPVPPPRRKRAVALSESNISAALASLADKTFXQPAVSSDSGAAFSTPTETSEPDPIIVDDKSDDGSYSSMPPLEGEPGDPDLTSDSWSTVSGSEDVVCCSMSYSWTGALVTPCAAEETKLPINPLSNSLLRHHNMVYSTTSRSAATRQKKVTFDRMQVVDSHYNXVLKEIKAQASTVKARLLTVEEACNLTPPHSARSKFGYGAKEVRSHTRKAINHINSVWEDLREDNTTPIPTTIMAKNEVFSVTPEKGGKKSARLIVYPDLGVRVCEKRALYDAVKQLSLAVMGTSYGFQYSPSQRVEFLLNAWRSKKTPMGFSYDTRCFDSTVTERDIRVEEEVYQCCDLEPEARKVISALTERLYVGGPMYNSRGDLCGIRRCRASGVFTTSFGNTLTCYLKANAAIRAAGLKDCTMLVCGDDLVVIAESDGVEEDKRALGAFTEAMTRYSAPPGDAPQPAYDLELITSCSSNVSVAHDGTGKRVYYLTRNPETPLARAAWETARHTPVNSWLGNIIIYAPTIWVRMVLMTHFFSILQSQEALEKALDFDMYGVTYSITPLDLPAIIQRLHGLSAFTLHGYSPHELNRVAGSLRKLGVPPLRAWRHRARAVRAKLIAQGGRARICGIYLFNWAVKTKAKLTPLPAAAKLDLSSWFTVGAGGGDIYHSVSHARPRYLLLCLLLLSVGVGIFLLPAR

SEQ ID NO: 3 - protein, 4 aa, Hepatitis C virus
DEAD

(19) United States
(12) Patent Application Publication
(10) Pub. No.: US 2015/0072418 A1
(43) Pub. Date: Mar. 12, 2015

(54) HCV GENOTYPE 4D REPLICONS

(71) Applicant: GILEAD SCIENCES, INC., FOSTER CITY, CA (US)

(72) Inventors: HADAS DVORY-SOBOL, FOSTER CITY, CA (US); CHRISTY HEBNER, BELMONT, CA (US); HONGMEI MO, PALO ALTO, CA (US); SIMIN XU, PALO ALTO, CA (US)

(21) Appl. No.: 14/459,913

(22) Filed: Aug. 14, 2014

Related U.S. Application Data
(60) Provisional application No. 61/866,948, filed on Aug. 16, 2013.

(51) Int. Cl. C12N 15/86; C12N 7/00

(52) U.S. Cl. CPC C12N 15/86; C12N 7/00; C12N 2770/24221; C12N 2770/24243
USPC 435/370; 435/320.1; 435/325

(57) ABSTRACT

Replicons of genotype 4d hepatitis C virus (HCV) are provided. These replicons contain adaptive mutations giving rise to the HCV's capability to replicate in vitro. Methods of preparing genotype 4d replicons and methods of using these replicons to screen antiviral agents are also provided.

### CROSS REFERENCE TO RELATED APPLICATION

This application claims the benefit under 35 U.S.C. §119(e) of U.S. Provisional Applications Ser. No. 61/866,948 filed Aug. 16, 2013, the content of which is incorporated by reference in its entirety into the present disclosure.

### FIELD OF THE DISCLOSURE

The disclosure is directed to hepatitis C replicons of genotype 4d and methods of preparing and using the replicons.

### STATE OF THE ART

Chronic hepatitis C virus (HCV) infection remains a significant global health burden with an estimated 160 million people infected worldwide. The current standard of care is 24 to 48 week courses of pegylated interferon plus ribavirin. Due to the partial efficacy and poor tolerability of this regimen, the discovery and development of new antiviral agents has been intensely pursued. Recently, these efforts have culminated in the FDA approval of two NS3 protease inhibitors (boceprevir and telaprevir) for use in combination with pegylated interferon and ribavirin for the treatment of chronic genotype 1 HCV infection. Many other inhibitors are in advanced clinical development, however, the majority are being developed to treat genotype 1 infections.

HCV is a positive-strand RNA virus that exhibits extraordinary genetic diversity. Six major genotypes (i.e. genotype 1-6) along with multiple subtypes (e.g. genotype 1a, 1b, 1c etc.) have been reported. Genotypes 1, 2 and 3 have worldwide distributions. Genotypes 1a or 1b are generally predominant in North America, South America, Europe and Asia. However, genotypes 2 and 3 are common and can constitute 20 to 50% of infections in many of these areas. Genotype 4a is the predominant in the Middle East and many African countries; up to 15% of the population of Egypt is infected with HCV and 93% of infections are genotype 4. Genotype 5 is prevalent in South Africa, while Genotype 6 is most common in Asia. Although most continents and countries have a “dominant” genotype, infected populations are almost universally made up of a mixture of multiple genotypes. Furthermore, the geographical distribution and diversity (epidemiology) of HCV infection is continuously evolving, due to large-scale immigration and widespread intravenous drug use. For instance, genotype 4a has noticeably spread into central and northern Europe. This presents a clinical challenge, since it is well documented that individual genotypes respond differently to both direct antivirals and immunomodulatory therapies, including the current standard of care.

HCV replicons are self-replicating RNA sequences derived from the HCV genome and have served as workhorses both for molecular virology studies and drug discovery. To date, replicons have been established from two genotypes and three subtypes (genotypes 1a, 1b and 2a). These replicons have been crucial in multiple aspects of drug discovery and development including the identification of novel inhibitor classes, the optimization of clinical candidates and the characterization of clinical resistance. Recently, there has been increasing interest in developing next-generation drugs that are active against all major HCV genotypes. Ideally, the approval of “pan-genotypic” drugs and regimens will greatly simplify the treatment of HCV.

A key step in the pursuit of pan-genotypic treatment regimens will be the development of in vitro tools that allow the study of all major genotypes and subtypes. Replicons derived from sequences of additional major genotypes are needed.

### SUMMARY

It has been discovered, unexpectedly, that clonal cell lines stably replicating genotype 4d replicons were obtained by electroporating in vitro transcribed subgenomic 4d RNA into HCV permissive cell lines. Adaptive mutations have been identified from these clones, as compared to the wild-type virus. When these mutations were engineered by site-directed mutagenesis and introduced into the cell lines, HCV genotype 4d replications ensued.

These adaptive mutations for genotype 4d were located in NS3 (E176G, A240V), NS4A (Q34R) or NS5A (S232G or S232I). It is noted that the numbering of these amino acid positions are relative to the starting location of each protein, and is independent of particular HCV 4d strains, as further explained below. The establishment of robust genotype 4d replicon systems provides powerful tools to facilitate drug discovery and development efforts.

Accordingly, one embodiment of the present disclosure provides an isolated genotype 4d hepatitis C viral (HCV) RNA construct that is capable of replication in a eukaryotic cell. In one aspect, the RNA sequence comprises a 5′NTR, an internal ribosome entry site (IRES), sequences encoding one or more of NS3, NS4A, NS4B, NS5A or NS5B, and a 3′NTR.

In one aspect, the construct comprises one or more adaptive mutations (or simply “mutations”) in NS3, NS4A, or NS5A. Non-limiting examples include NS3 (E176G, A240V), NS4A (Q34R) and/or NS5A (S232G/I). It is also contemplated that the construct includes at least two, or alternatively three or four adaptive mutations. In one aspect, the construct includes NS4A (Q34R) and/or NS5A (S232G/I) but can be wild-type at positions NS3 (E176 and A240). In one aspect, the adaptive mutations come from different genes. In some aspects, the construct is a subgenomic or full-length HCV replicon.

Moreover, DNA that transcribes to the RNA construct, viral particles that include the RNA construct, and cells containing such DNA or RNA are also provided.

Also provided, in one embodiment, are individual NS3, NS4A or NS5A proteins that include one or more of the corresponding adaptive mutations. Polynucleotides encoding these proteins and antibodies that specifically recognize the proteins are also provided.

In another embodiment, the present disclosure provides an isolated cell comprising a genotype 4d hepatitis C viral (HCV) RNA that replicates in the cell. In one aspect, there is an absence, in the cell, of a DNA construct encoding the RNA. In another aspect, the cell comprises at least 10 copies, or alternatively at least about 100, 500, 1000, 2000, 5000, 10,000, 1×10⁵, 1×10⁶, 1×10⁷, 1×10⁸ or 1×10⁹ copies of the RNA. In any of such aspects, the RNA can be a subgenomic HCV sequence or a full-length HCV sequence and can include one or more of the adaptive mutations described above.

In one aspect, the cell is a mammalian cell which can be, for instance, a hepatoma cell, in particular a Huh7 1C cell.

Methods of improving the capability of a genotype 4d HCV viral RNA to replicate in a eukaryotic cell are also provided, comprising one or more of (a) substituting residue 34 of NS4A with an arginine, (b) substituting residue 176 of NS3 with glycine, (c) substituting residue 240 of NS3 with valine, and/or (d) substituting 232 of NS5A with glycine or isoleucine. In one aspect, the method entails (a) substituting residue 34 of NS4A with an arginine, and/or (b) substituting residue 240 of NS3 with valine, without modifying amino acid residues at NS3 (E176 and A240).

Still provided, in one embodiment, is a method of identifying an agent that inhibits the replication or activity of a genotype 4d HCV, comprising contacting a cell of any of the above embodiments with a candidate agent, wherein a decrease of replication or a decrease of the activity of a protein encoded by the RNA indicates that the agent inhibits the replication or activity of the HCV. Alternatively, the method comprises contacting the lysate of a cell of any of the above embodiments with a candidate agent, wherein a decrease of the activity of a protein encoded by the RNA indicates that the agent inhibits the activity of the HCV.

### BRIEF DESCRIPTION OF THE DRAWINGS

The disclosure is best understood from the following detailed description when read in conjunction with the accompanying drawings. Included in the drawings are the following

### FIGURES

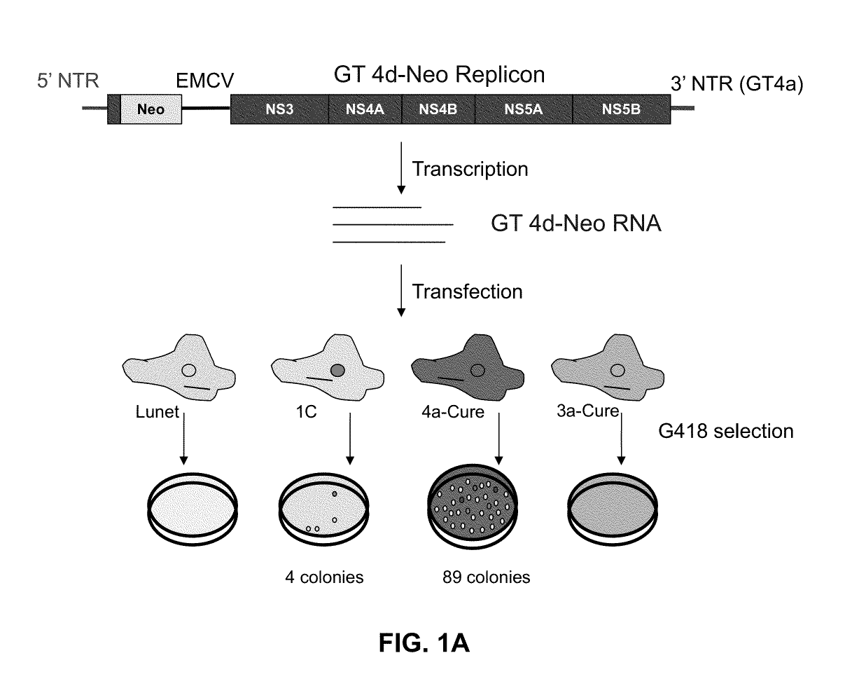

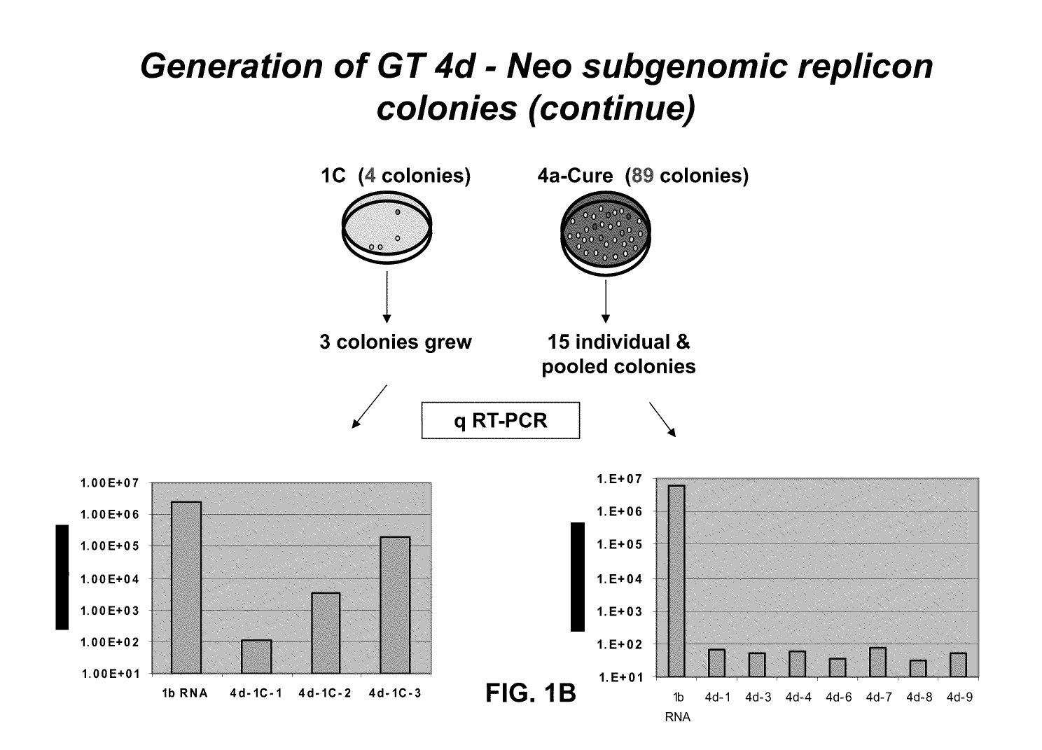

FIG. 1A-B present a schematic diagram of the process of generation of GT 4d-Neo subgenomic replicon colonies.

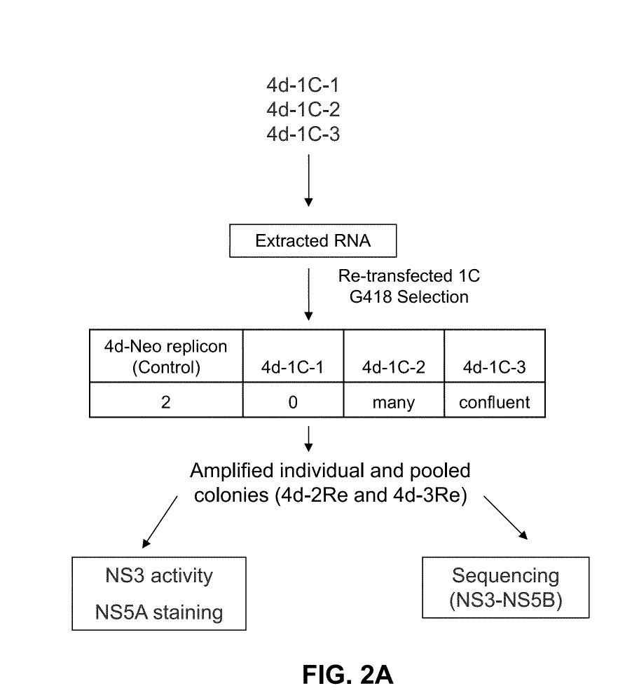

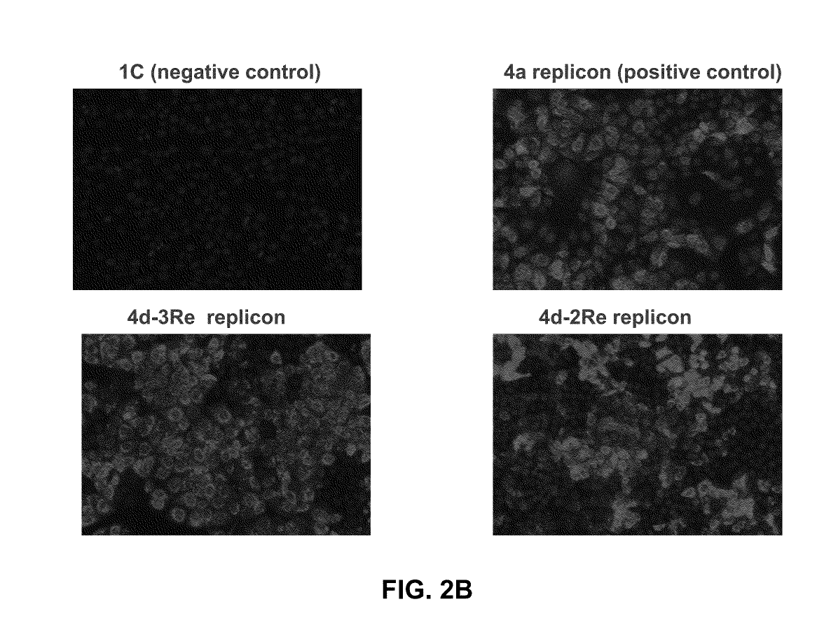

FIG. 2A shows the process of retransfection of total cellular RNA extracted from colonies of 4d-1C-1, 4d-1C-2 and 4d-1C-3 into cells for confirmation and sequencing. FIG. 2B presents images conforming the expression of HCV GT 4d-Neo replicon with NS5A staining NS5A expression was higher in 4d-3Re than in 4d-2Re. NS5A staining correlated with NS3 activity of 4d-3Re and 4d-2Re

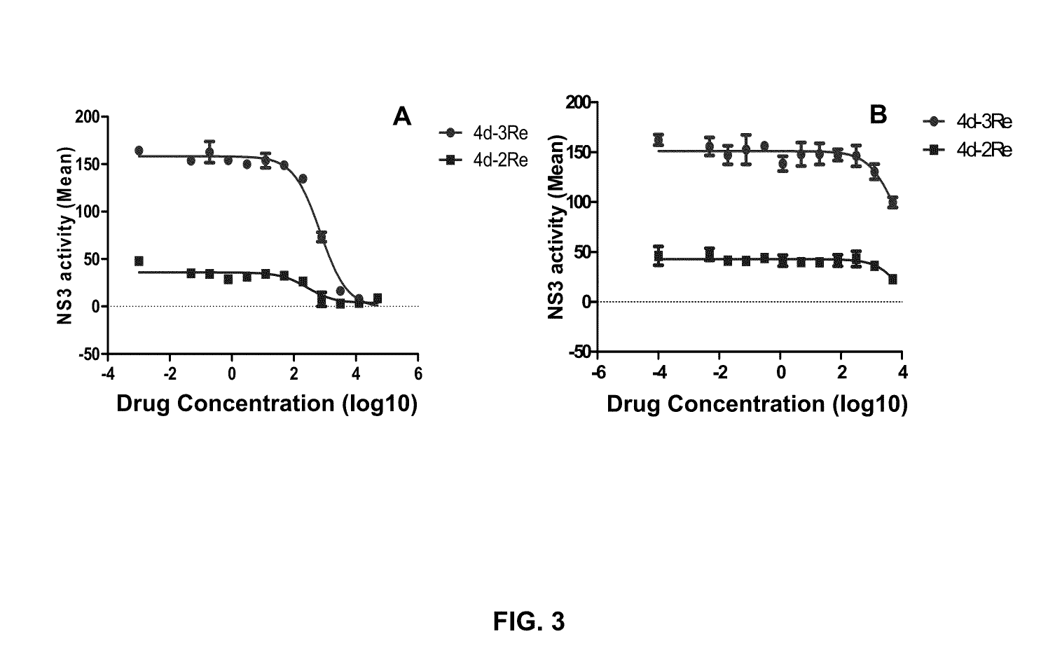

FIG. 3A-B include charts to show that 4d-3Re and 4d-2Re showed dose dependent inhibition of NS3 activity by Compound A (3A), and a slight inhibition at high concentration of Compound B (3B).

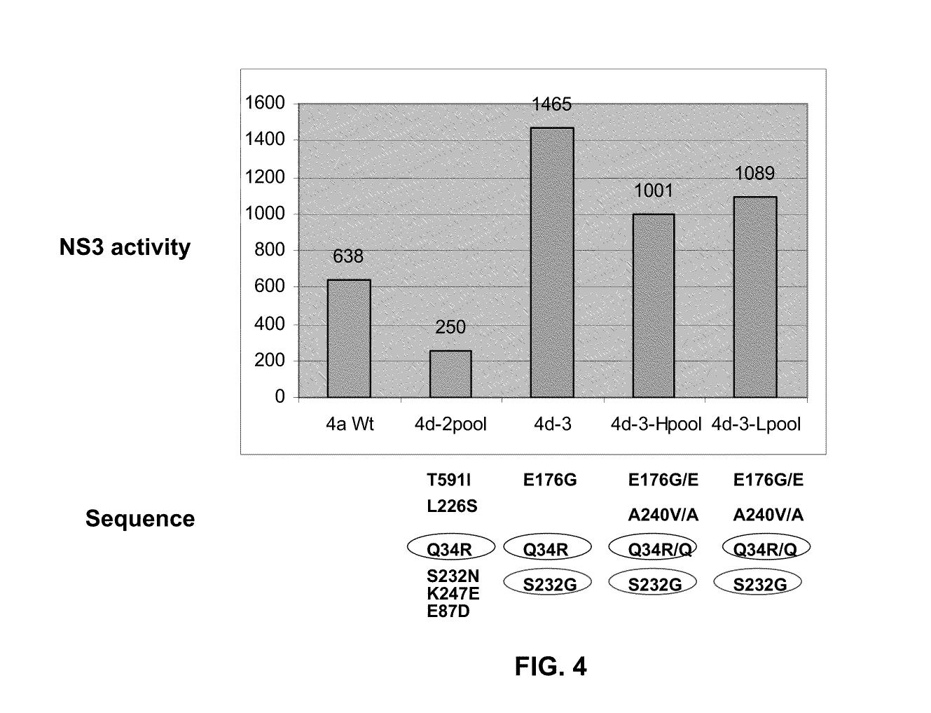

FIG. 4 shows comparison of replication levels among GT-4d-Neo colonies.

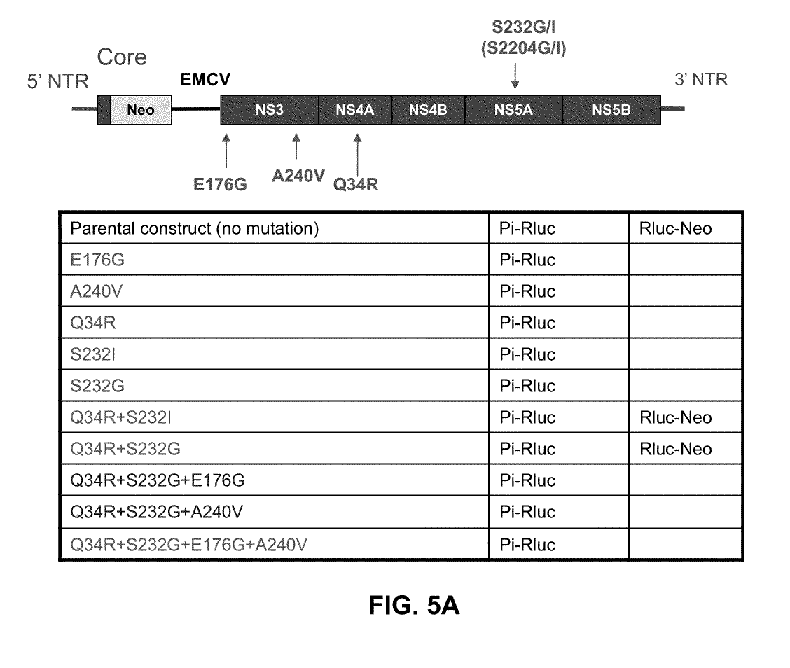

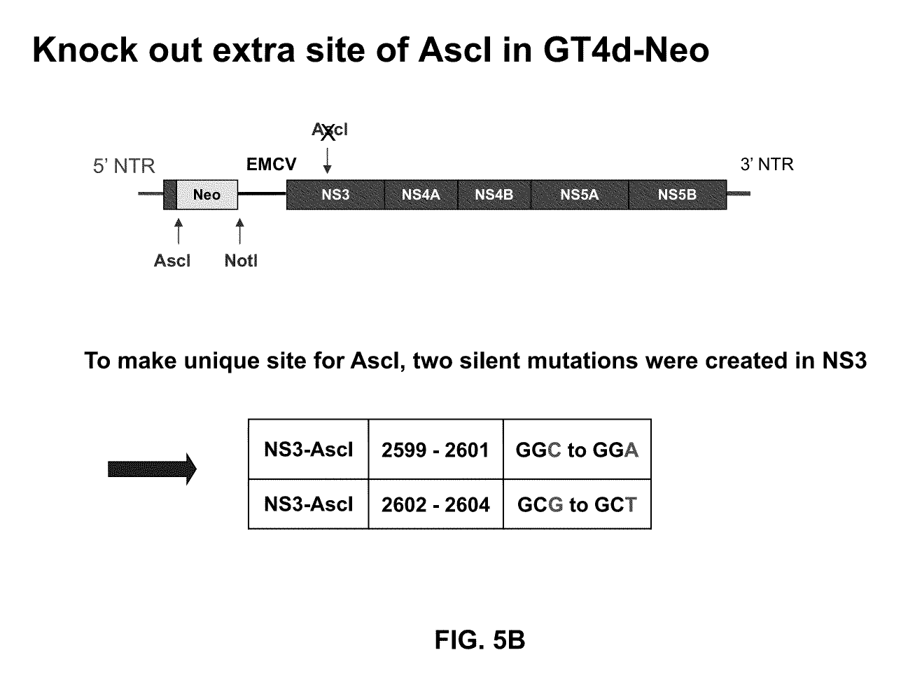

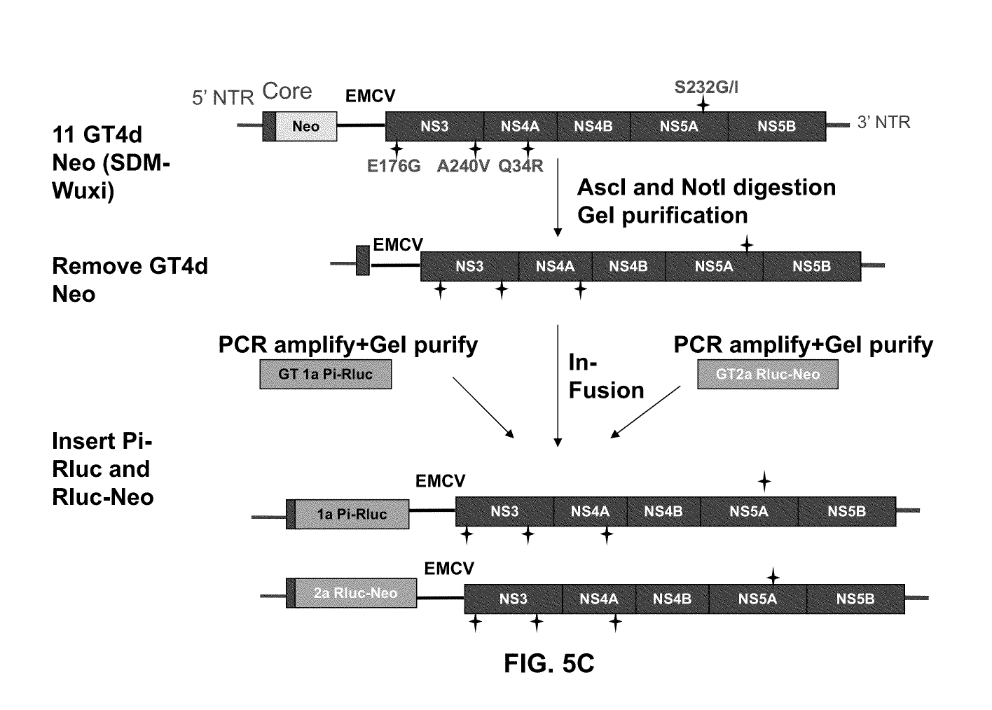

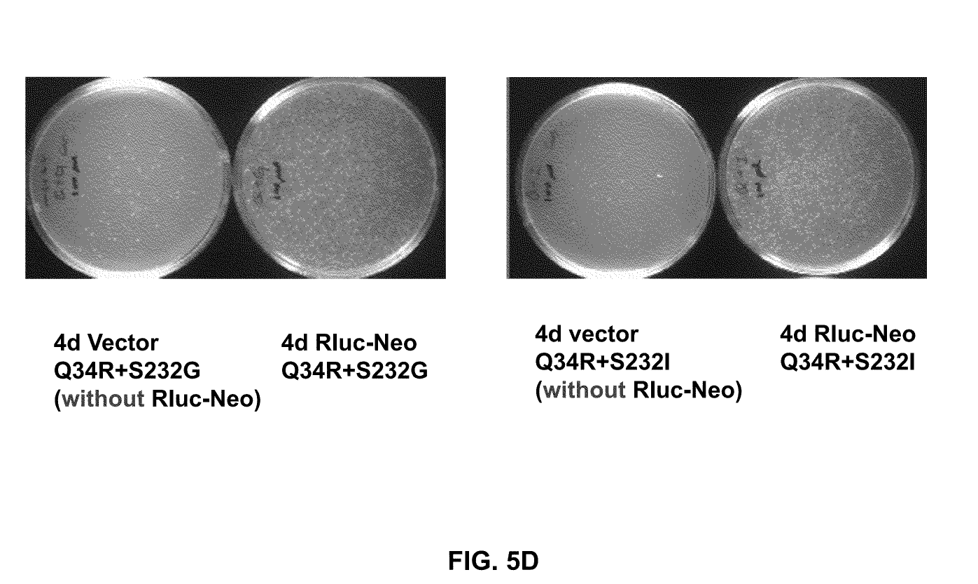

FIG. 5A-D show the design and preparation of GT4d Pi-Rluc and Rluc-Neo constructs. In particular, FIG. 5D shows the colonies of Rluc-Neo construct (replaced the Neo) generated by in-fusion method.

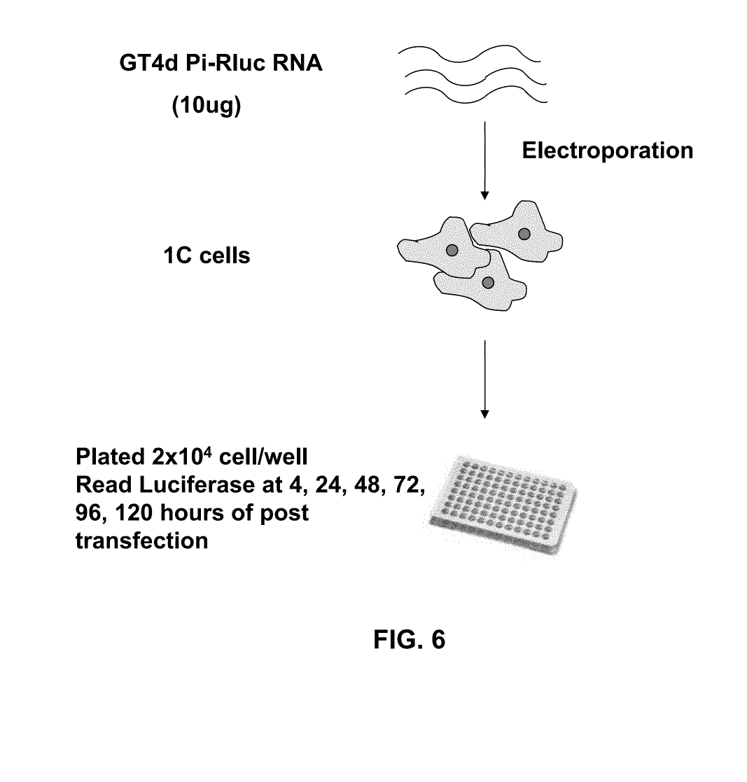

FIG. 6 shows the generation of replication time course for adaptive mutations in GT4d Pi-Rluc replicon.

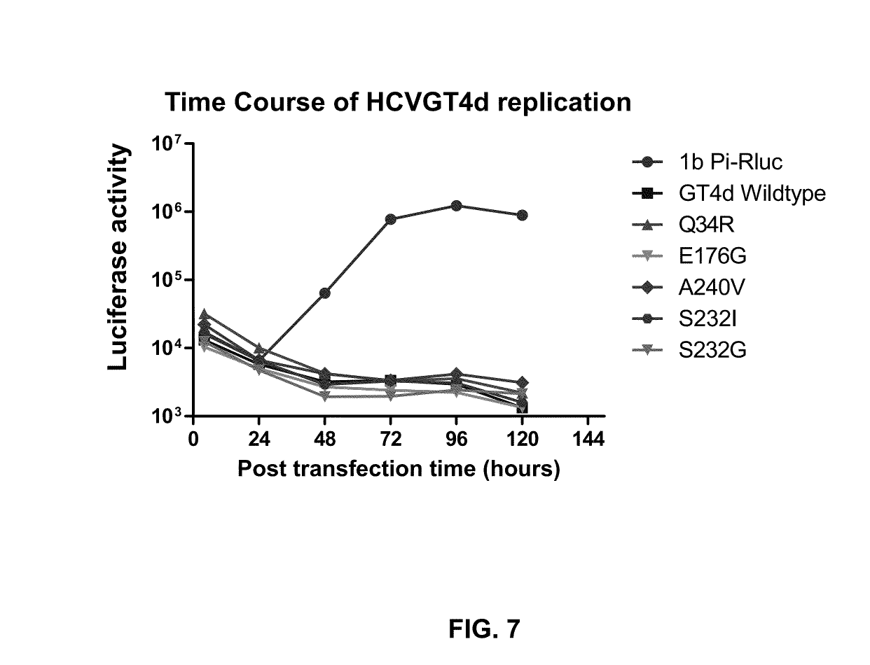

FIG. 7 shows the replication curves of 4d Pi-Rluc replicons carrying single adaptive mutations.

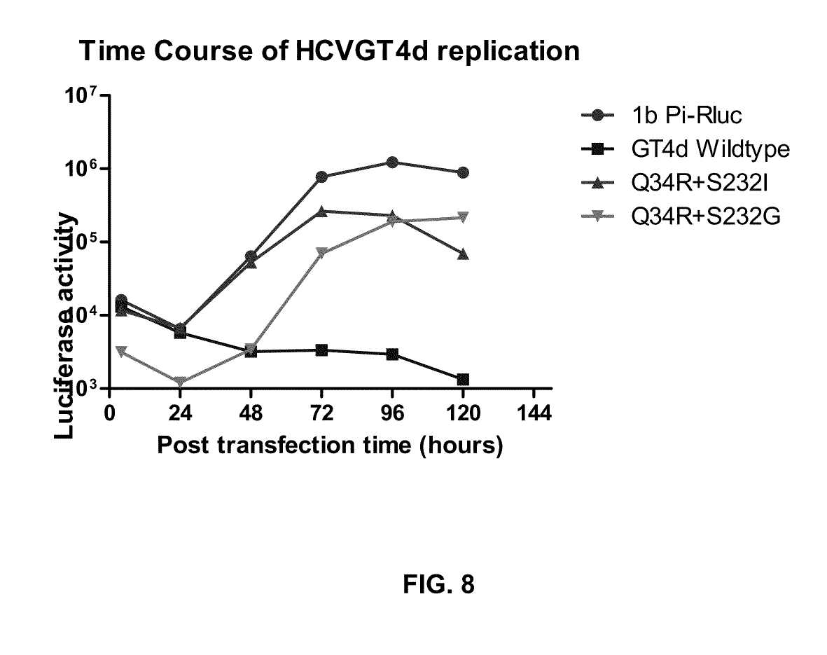

FIG. 8 shows the replication curves of 4d Pi-Rluc replicons carrying double adaptive mutations (Q34R+S232I or Q34R+S232G).

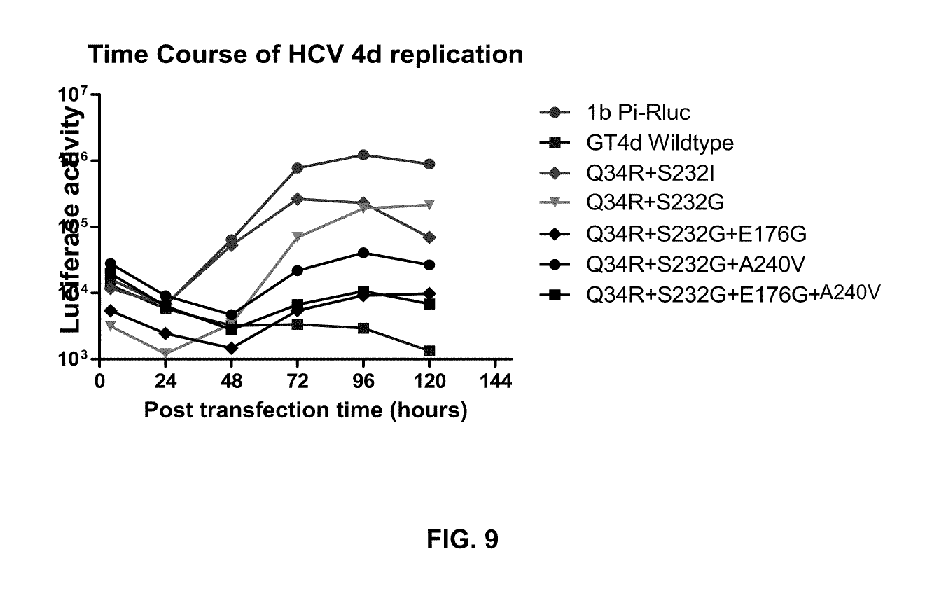

FIG. 9 shows the replication curves of 4d Pi-Rluc replicons carrying double, triple and all four adaptive mutations.

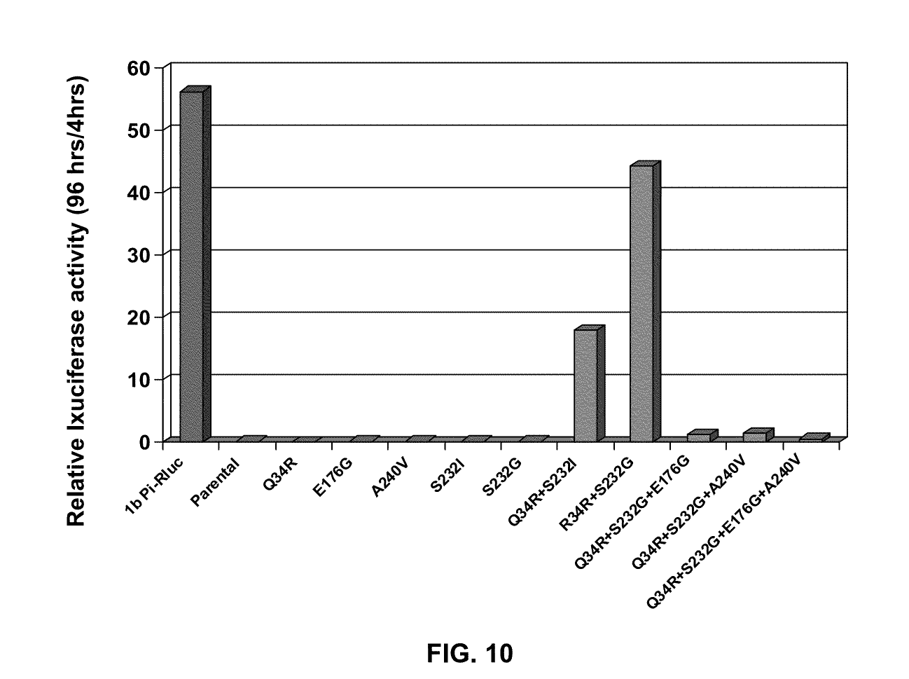

FIG. 10 compares the replication capacity of different replicons at 96 hours post transfection.

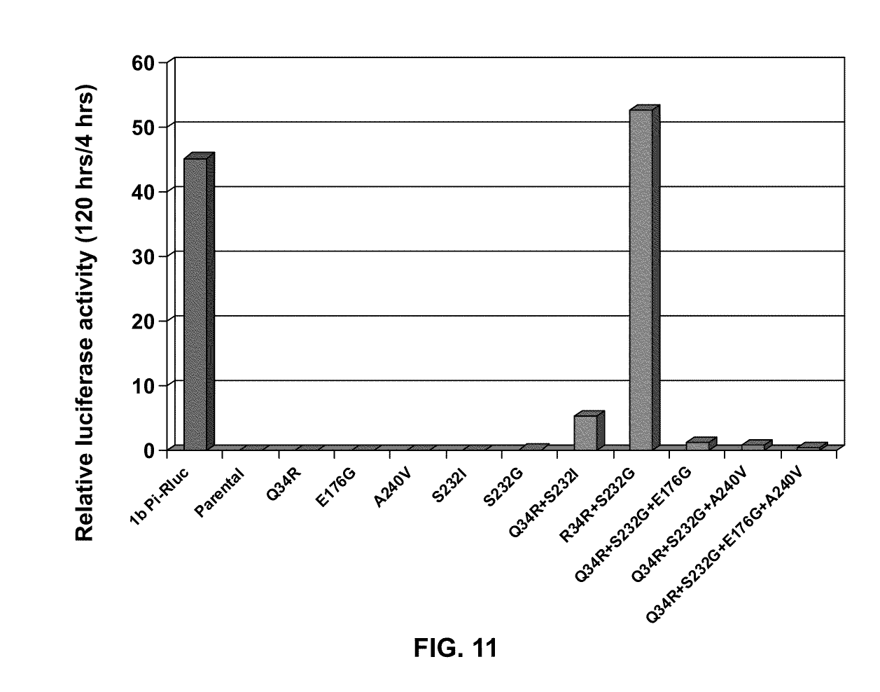

FIG. 11 compares the replication capacity of different replicons at 120 hours post transfection.

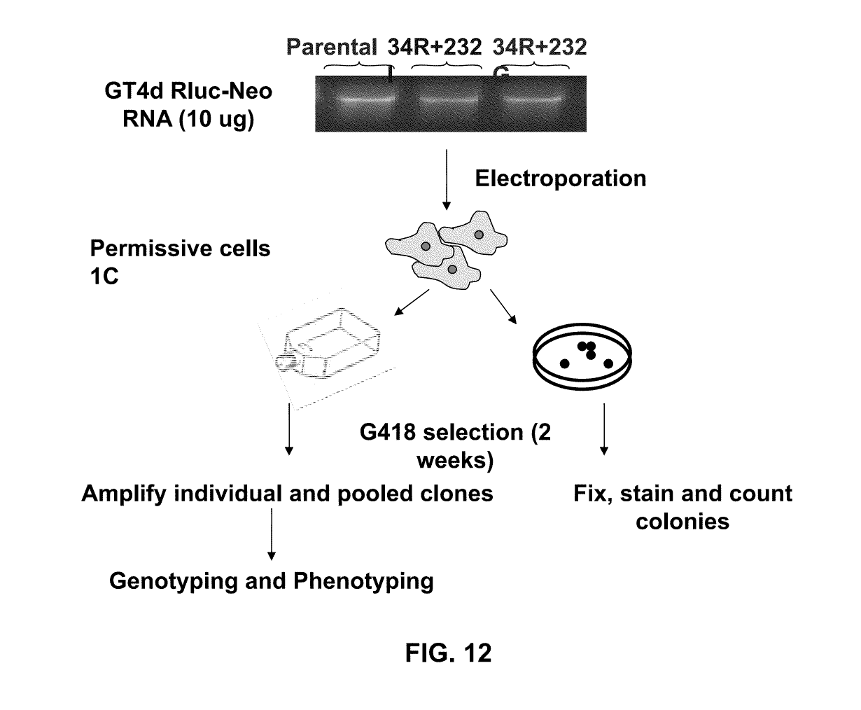

FIG. 12 illustrates the process of generation of stable GT4d Rluc-neo subgenomic replicons.

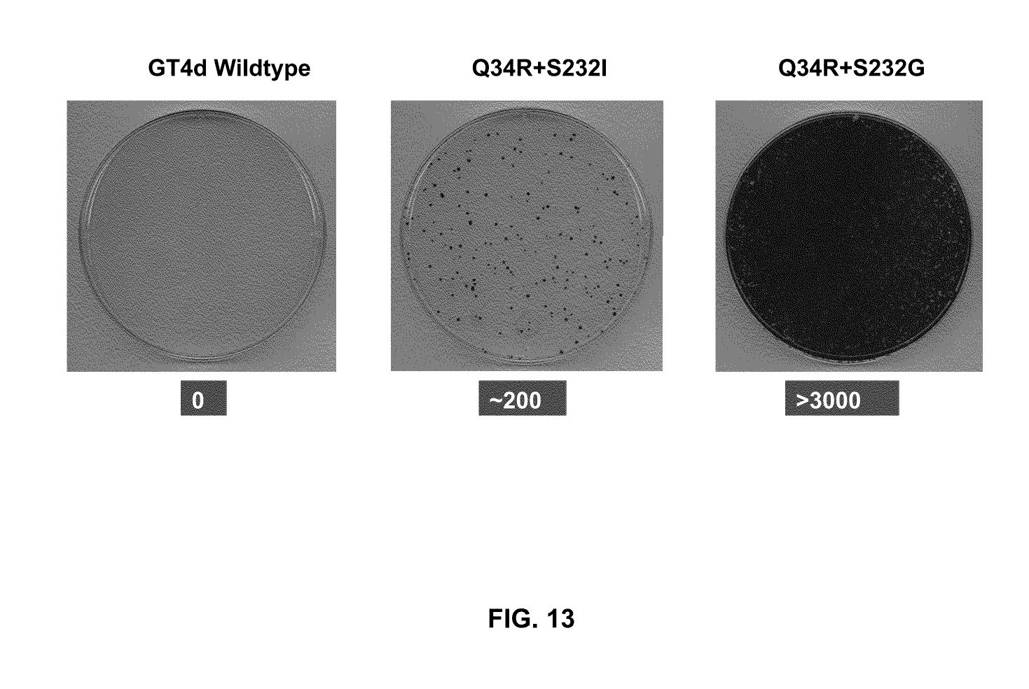

FIG. 13 shows the colony formation efficiency for different 4d Rluc-Neo replicons.

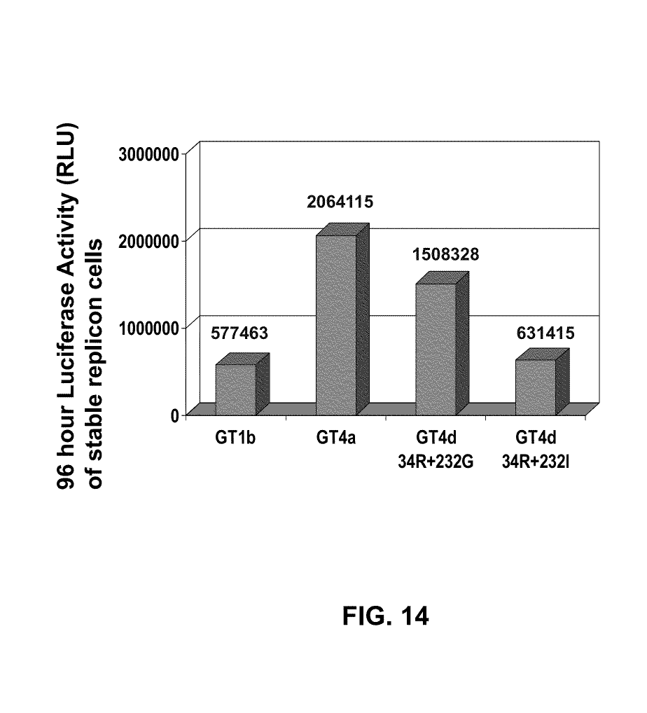

FIG. 14 compares the luciferse activity of stable replicon cells of the double-mutation GT4d replicons to GT4a and GT1b replicons.

### DETAILED DESCRIPTION

Prior to describing this disclosure in greater detail, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a thread” includes a plurality of threads.

### 1. DEFINITIONS

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The term “protein” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Single letter and three letter abbreviations of the naturally occurring amino acids are listed below. A peptide of three or more amino acids is commonly called an oligopeptide if the peptide chain is short. If the peptide chain is long, the peptide is commonly called a polypeptide or a protein.

| 1-Letter | 3-Letter | Amino Acid |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptohan |
| R | Arg | L-arginine |
| D | Asp | L-aspartic acid |
| N | Asn | L-asparagine |
| C | Cys | L-cysteine |

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention. In one embodiment, the homologous peptide is one that shares the same functional characteristics as those described, including one or more of the adaptive mutations.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/blast/Blast.cgi, last accessed on Jul. 15, 2011. Biologically equivalent polynucleotides are those having the specified percent homology and encoding a polypeptide having the same or similar biological activity.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells, or alternatively from a prokaryotic cells as described above. Non-limiting examples include simian, bovine, porcine, murine, rats, avian, reptilian and human.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The antibodies can be polyclonal or monoclonal and can be isolated from any suitable biological source, e.g., murine, rat, sheep and canine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “isolated” as used herein refers to molecules or biological or cellular materials being substantially free from other materials or when referring to proteins or polynucleotides, infers the breaking of covalent bonds to remove the protein or polynucleotide from its native environment. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. The term “isolated” is also used herein to refer to cells or tissues that are isolated from other cells or tissues and is meant to encompass both cultured and engineered cells or tissues.

Hepatitis C virus or “HCV” is a small (55-65 nm in size), enveloped, positive-sense single-stranded RNA virus of the family Flaviviridae. Hepatitis C virus is the cause of hepatitis C in humans. The hepatitis C virus particle consists of a core of genetic material (RNA), surrounded by an icosahedral protective shell of protein, and further encased in a lipid (fatty) envelope of cellular origin. Two viral envelope glycoproteins, E1 and E2, are embedded in the lipid envelope.

Hepatitis C virus has a positive sense single-stranded RNA genome. The genome consists of a single open reading frame that is 9600 nucleotide bases long. This single open reading frame is translated to produce a single protein product, which is then further processed to produce smaller active proteins.

At the 5′ and 3′ ends of the RNA are the UTR, that are not translated into proteins but are important to translation and replication of the viral RNA. The 5′ UTR has a ribosome binding site (IRES—Internal ribosome entry site) that starts the translation of a very long protein containing about 3,000 amino acids. This large pre-protein is later cut by cellular and viral proteases into the 10 smaller proteins that allow viral replication within the host cell, or assemble into the mature viral particles.

Structural proteins made by the hepatitis C virus include Core protein, E1 and E2; nonstructural proteins include NS2, NS3, NS4A, NS4B, NS5A, and NS5B.

Based on genetic differences between HCV isolates, the hepatitis C virus species is classified into six genotypes (1-6) with several subtypes within each genotype (represented by letters). Subtypes are further broken down into quasispecies based on their genetic diversity. The preponderance and distribution of HCV genotypes varies globally. For example, in North America, genotype 1a predominates followed by 1b, 2a, 2b, and 3a. In Europe, genotype 1b is predominant followed by 2a, 2b, 2c, and 3a. Genotypes 4 and 5 are found almost exclusively in Africa. Genotype is clinically important in determining potential response to interferon-based therapy and the required duration of such therapy. Genotypes 1 and 4 are less responsive to interferon-based treatment than are the other genotypes (2, 3, 5 and 6). Duration of standard interferon-based therapy for genotypes 1 and 4 is 48 weeks, whereas treatment for genotypes 2 and 3 is completed in 24 weeks.

Sequences from different HCV genotypes can vary as much as 33% over the whole viral genome and the sequence variability is distributed equally throughout the viral genome, apart from the highly conserved 5′ UTR and core regions and the hypervariable envelope (E) region.

HCV genotypes can be identified with various methods known in the art. PCR-based genotyping with genotype-specific primers was first introduced in 1992, in particular with primers targeting the core region. Commercial kits (e.g., InnoLipa® by Innogenetics (Zwijindre, Belgium)) are also available. Direct sequencing, in the vein, can be used for more reliable and sensitive genotyping.

Serologic genotyping uses genotype-specific antibodies and identifies genotypes indirectly. Two commercially available serologic genotyping assays have been introduced, including a RIBA SIA assay from Chiron Corp. and the Murex HCV serotyping enzyme immune assay from Nurex Diagnostics Ltd.

Sequences of genotype 4d HCV have been identified. For instance, GenBank accession # DQ516083 represents a subtype 4d isolate 24 polyprotein gene. Further discussion of the genotype 4d and their sequences are clinical impacts can be found at Zein Clin. Microbiol. Rev. 13(2):223-35 (2000).

Despite the sequence variability between different genotypes of HCV or even within a particular genotype, there is consensus in the numbering of amino acid residues and nucleotide bases, and thus the numbering does not depend on a particular strain. Such a standard numbering system is described in, for instance, Kuiken et al., “A Comprehensive System for Consistent Numbering of HCV Sequences, Proteins and Epitopes,” Hepatology, 44(5):1355-61 (2006) and Kuiken and Simmonds “Nomenclature and Numbering of the Hepatitis C Virus,” Hengli Tang (ed.), Hepatitis C: Methods and Protocols, Second Edition, vol. 510:33-53 (2009).

The standard numbering system, for both nucleotides and amino acid sequences, uses the full-length genome sequence of isolate H77 (accession number AF009606) as a reference. The numbering can be absolute, which starts at the first nucleotide of the RNA, or the first amino acid of the core protein, and continue through the end of the RNA or NS5B, or relative, which starts over at every protein, as shown in the table below, adapted from Kuiken et al. (2009).

| | Nucleic acid | Nucleic acid | Amino acid | Amino acid | |
|---|---|---|---|---|---|
| | absolute | relative | absolute | relative | |
| Region | numbering | numbering | numbering | numbering | Description |
| | 1-341 | 1-341 | | | 5 untranslated region |
| Core | 342-914 | 1-573 | 1-191 | 1-191 | Core protein |
| | 915-1490 | 1-576 | 192-383 | 1-192 | Envelope glycoprotein 1 |
| E2 | 1491-2579 | 1-1089 | 384-746 | 1-363 | Envelope glycoprotein 2 |
| | 2580-2768 | 1-189 | 747-809 | 1-63 | Putative ion channel |
| NS2 | 2769-3419 | 1-651 | 810-1026 | 1-217 | Autoprotease |
| NS3 | 3420-5312 | 1-1893 | 1027-1657 | 1-631 | Serine protease and |
| | | | | | RNA-dependent RNA |
| | | | | | helicase |
| NS4A | 5313-5474 | 1-162 | 1658-1711 | 1-54 | NS3 cofactor |
| | 5475-6257 | 1-783 | 1712-1972 | 1-261 | NS4B protein |
| NS5A | 6258-7601 | 1-1344 | 1973-2420 | 1-448 | NS5A phosphoprotein |
| NS5B | 7602-9377 | 1-1776 | 2421-3011 | 1-591 | RNA-dependent RNA |
| | | | | | polymerase |
| 3UTR | 9378-9646 | 1-269 | | | 3 untranslated region |

The term “replicon” refers to a DNA molecule or RNA molecule, or a region of DNA or RNA, that replicates from a single origin of replication. For most prokaryotic chromosomes, the replicon is the entire chromosome. In some aspects, a replicon refers to a DNA or RNA construct that replicates in a cell in vitro. In one aspect, a replicon can replicate to produce at least about 10, or alternatively, at least about 100, 500, 1000, 2000, 5000, 10,000, 1×10⁵, 1×10⁶, 1×10⁷, 1×10⁸ or 1×10⁹ copies of the replicon in a cell in vitro. Alternatively, a replicon's replication efficiency can be measured by producing certain amount of viral RNA in total RNA that includes cellular RNA. In one aspect, a replicon can produce at least about 1000, 1×10⁴, 1×10⁵, 1×10⁶, 1×10⁷, 1×10⁸, 1×10⁹, 1×10¹⁰, 1×10¹¹, or 1×10¹² copies of the replicon per microgram of total RNA or cellular RNA.

A “subgenomic” HCV sequence refers to a HCV sequence that does not include all sequences of a wild-type HCV. In one aspect, a subgenomic HCV or a subgenomic HCV replicon does not include the E1, E2 or C regions. In another aspect, a subgenomic HCV or a subgenomic HCV replicon includes all or part of the 5′ UTR, NS3, NS4A, NS4B, NS5A, NS5B and 3′ UTR sequences. In contrast, a “full-length” or “full genome” HCV or HCV replicon includes E1, E2 and C regions. In some aspects, both a subgenomic and a full-length HCV replicon can include one or more of a reporter gene (e.g., luciferase), a marker gene (e.g., Neo), and an IRES (e.g., EMCV IRES) sequence.

A virus particle (or virion) consists of the genetic material made from either DNA or RNA of a virus and a protein coat that protects the genetic material. In one aspect, an envelope of lipids surrounds the protein coat when they are outside a cell.

The term “adaptive mutation” of a HCV replicon of a certain genotype refers to a mutation, as compared to a wild-type HCV sequence of the genotype, that enables the wild-type replicon to replicate in a cell, in particular in a eukaryotic cell such as a mammalian cell and in vitro, or enhances a HCV replicon's ability to replicate. It is contemplated that an adaptive mutation can favorably influence assembly of the replicase complex with host cell-specific protein, or alternatively promote interactions of the protein that includes the adaptive mutation (e.g., NS3, NS4A, NS4B, NS5A etc) with cellular proteins involved in host cell antiviral defenses.

A “reporter gene” refers to a gene that can be attached to a regulatory sequence of another gene of interest in cell culture, animals or plants, to facilitate identification of this other gene. Reporter genes are often used as an indication of whether a certain gene has been taken up by or expressed in the cell or organism population. Non-limiting examples of reporter gene include the luciferase gene and the green fluorescent protein gene.

A “marker gene” or “selectable marker” refers to a gene that protects the organism from a selective agent that would normally kill it or prevent its growth. One non-limiting example is the neomycin phosphotransferase gene (Neo), which upon expression confers resistance to G418, an aminoglycoside antibiotic similar in structure to gentamicin B1.

Sofosbuvir (brand name Sovaldi®) is a drug used to treat hepatitis C infection. In combination with other therapies, Sofosbuvir inhibits the RNA polymerase that the hepatitis C virus uses to replicate its RNA. The chemical name of Sofosbuvir is isopropyl (2S)-2-[[[(2R,3R,4R,5R)-5-(2,4-dioxopyrimidin-1-yl)-4-fluoro-3-hydroxy-4-methyl-tetrahydrofuran-2-yl]methoxy-phenoxy-phosphoryl]amino]propanoate.

### HCV Genotype 4d Replicon Constructs

The present disclosure relates, in general, to the unexpected discovery that clonal cell lines stably replicating genotype 4d replicons can be obtained by eletroporating in vitro transcribed 4d RNA into HCV permissive cell lines. From the clonal cells, adaptive mutations are then identified.

These adaptive mutations were located in NS3 (E176G, A240V), NS4A (Q34R) or NS5A (S232G/I). The numbering of the amino acid residues in the present disclosure is relative to each individual protein, except for 5232 for which both relative numbering (232) and absolute numbering (2204) are used. Further, such numberings are strain-independent and use a standard numbering system as noted in Kuiken et al. (2006) and Kuiken and Simmonds (2009). Moreover, each mutation noted in the disclosure is relative to the wild-type HCV genotype 4d sequence, exemplified by GT4d isolate QC382 accession number FJ462437 (SEQ ID NO: 1).

Identification of these mutations suggests that these mutations contribute to the HCV's capability to replicate in cells in vitro, a phenomenon not observed with wild-type HCV genotype 4d RNA. Such contribution has then been confirmed by engineering the mutations, by site-directed mutagenesis, into genotype 4d RNA and introducing them into the cell lines. Genotype 4d HCV RNA, with such mutations, successfully replicated in the cell lines. Therefore, the Applicant has demonstrated that the Applicant has prepared HCV genotype 4d replicons capable of replication in vitro and has identified adaptive mutations leading to such capabilities.

Accordingly, in one embodiment, the present disclosure provides a genotype 4d hepatitis C viral (HCV) RNA is capable of replication in a host cell. In one aspect, the replication is in vitro. In another aspect, the replication is productive. In another aspect, the cell is a eukaryotic cell such as a mammalian cell or a human cell. In yet another aspect, the cell is a hepatoma cell. In some aspects, the RNA can replicate to produce at least 10 copies of the RNA in a cell. In another aspect, the number of copies is at least about 100, 500, 1000, 2000, 5000, 10,000, 1×10⁵, 1×10⁶, 1×10⁷, 1×10⁸ or 1×10⁹.

The HCV RNA can be a subgenomic HCV sequence. It is specifically contemplated that a full-length HCV replicon containing one or more of such adaptive mutations is also capable to replicate. Still further, an entire HCV virus of the corresponding genotype containing the adaptive mutation(s) would be infectious and capable to replicate. In any such case, RNA can include one or more of 5′NTR, an internal ribosome entry site (IRES), sequences encoding NS3, NS4A, NS4B, NS5A and NS5B, and a 3′NTR. In one aspect, the RNA includes, from 5′ to 3′ on the positive-sense nucleic acid, a functional HCV 5′ non-translated region (5′NTR) comprising an extreme 5′-terminal conserved sequence; an HCV polyprotein coding region; and a functional HCV 3′ non-translated region (3′NTR) comprising an extreme 3′-terminal conserved sequence.

Non-limiting examples of adaptive mutation for genotype 4d also include NS3 (E176G, A240V), NS4A (Q34R) or NS5A (S232G/I). In some embodiments, the replicon includes either or both of NS4A (Q34R) and NS5A (S232G/I). In some embodiments, the replicon does not include mutations (i.e., is wild-type) at NS3 (E176 and A240). It is further contemplated that, for any embodiment of the present disclosure, the Q34R mutation can be substituted with a Q34K mutation.

Also contemplated are that the HCV RNA can be a RNA sequence that has at least about 75%, or about 80%, 85%, 90%, 95%, 98%, 99%, or about 99.5% sequence identity to any of the disclosed sequences, so long as it retains the corresponding adaptive mutation(s) and/or activities.

Also provided is a genotype 4d hepatitis C viral (HCV) RNA construct comprising a nuclei acid sequence of SEQ ID NO: 1 or a polynucleotide having at least 95% sequence identity to SEQ ID NO: 1, wherein the construct comprises nucleotides coding for an arginine residue 34 in NS4A and/or a glycine or isoleucine at residue 232 in NS5A.

SEQ ID NO: 1 provides the sequence for GT4d isolate QC382 (accession FJ462437) sequence, and the numbering of these residues are according to the genes within the sequence.

SEQ ID NO: 1
(GT4d isolate QC382 FJ462437)
ACCTGCTCTCTATGAGAGCAACACTCCACCATGAACCGCTCCCCTGTGAGGAACTACTGTCTTCACGCAGA
AAGCGTCTAGCCATGGCGTTAGTATGAGTGTTGTACAGCCTCCAGGACCCCCCCTCCCGGGAGAGCCATAG
TGGTCTGCGGAACCGGTGAGTACACCGGAATCGCCGGGATGACCGGGTCCTTTCTTGGATTAACCCGCTCA
ATGCCCGGAAATTTGGGCGTGCCCCCGCAAGACTGCTAGCCGAGTAGTGTTGGGTCGCGAAAGGCCTTGTG
GTACTGCCTGATAGGGTGCTTGCGAGTGCCCCGGGAGGTCTCGTAGACCGTGCACCATGAGCACGAATCCT
AAACCTCAAAGAAAAACCAAACGTAACACCAACGGCGCGCCAATGATTGAACAAGATGGATTGCACGCAGG
TTCTCCGGCCGCTTGGGTGGAGAGGCTATTCGGCTATGACTGGGCACAACAGACAATCGGCTGCTCTGATG
CCGCCGTGTTCCGGCTGTCAGCGCAGGGGCGCCCGGTTCTTTTTGTCAAGACCGACCTGTCCGGTGCCCTG
AATGAACTGCAGGACGAGGCAGCGCGGCTATCGTGGCTGGCCACGACGGGCGTTCCTTGCGCAGCTGTGCT
CGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCG
GCTACCTGCCCATTCGACCACCAAGCGAAACATCGCATCGAGCGAGCACGTACTCGGATGGAAGCCGGTCT
TGTCGATCAGGATGATCTGGACGAAGAGCATCAGGGGCTCGCGCCAGCCGAACTGTTCGCCAGGCTCAAGG
CGCGCATGCCCGACGGCGAGGATCTCGTCGTGACCCATGGCGATGCCTGCTTGCCGAATATCATGGTGGAA
AATGGCCGCTTTTCTGGATTCATCGACTGTGGCCGGCTGGGTGTGGCGGACCGCTATCAGGACATAGCGTT
GGCTACCCGTGATATTGCTGAAGAGCTTGGCGGCGAATGGGCTGACCGCTTCCTCGTGCTTTACGGTATCG
CCGCTCCCGATTCGCAGCGCATCGCCTTCTATCGCCTTCTTGACGAGTTCTTCTGAGCGGCCGCGTTGTTA
AACAGACCACAACGGTTTCCCTCTAGCGGGATCAATTCCGCCCCCCCCCCCTAACGTTACTGGCCGAAGCC
GCTTGGAATAAGGCCGGTGTGCGTTTGTCTATATGTTATTTTCCACCATATTGCCGTCTTTTGGCAATGTG
AGGGCCCGGAAACCTGGCCCTGTCTTCTTGACGAGCATTCCTAGGGGTCTTTCCCCTCTCGCCAAAGGAAT
GCAAGGTCTGTTGAATGTCGTGAAGGAAGCAGTTCCTCTGGAAGCTTCTTGAAGACAAACAACGTCTGTAG
CGACCCTTTGCAGGCAGCGGAACCCCCCACCTGGCGACAGGTGCCTCTGCGGCCAAAAGCCACGTGTATAA
GATACACCTGCAAAGGCGGCACAACCCCAGTGCCACGTTGTGAGTTGGATAGTTGTGGAAAGAGTCAAATG
GCTCTCCTCAAGCGTATTCAACAAGGGGCTGAAGGATGCCCAGAAGGTACCCCATTGTATGGGATCTGATC
TGGGGCCTCGGTGCACATGCTTTACATGTGTTTAGTCGAGGTTAAAAAAACGTCTAGGCCCCCCGAACCAC
GGGGACGTGGTTTTCCTTTGAAAAACACGATAATACCATGGCCCCTATCACTGCGTATGCGCAACAGACCC
GGGGGACGCTAGGCACCATAATCACAAGCCTCACCGGCAGAGATACCAACGAGAACTGCGGTGAAATCCAG
GTGCTGTCCACGGCGACGCAGTCTTTCTTGGGCAGTGCGATCAATGGCGTCATGTGGACGGTTTACCATGG
GGCGGGCAGCAAGACCATCAGCGGCCCGAAAGGACCGGTCAACCAGATGTACACCAATGTCGACCAAGACT
TGGTGGGCTGGCCCGCACCTCCAGGAGTGAAGTCCTTGGCCCCATGCACCTGTGGCTCGTCGGACCTGTTC
CTGGTCACCAGGCACGCCGACGTGGTGCCCGTGCGCAGAAGAGGCGACACTCGTGGCGCCCTCTTAAGCCC
TAGGCCGATTTCAACTCTTAAGGGATCATCCGGTGGGCCACTGTTGTGCCCCCTGGGTCACGTCGCCGGCA
TCTTCCGAGCCGCGGTGTGTACCCGGGGCGTGGCCAAAGCAGTGGACTTCGTACCGGTTGAATCTCTTGAA
ACCACCATGAGGTCTCCAGTATTCTCTGACAATTCCACTCCTCCTGCCGTGCCCCAGACTTACCAAGTAGC
CCACTTGCACGCGCCAACGGGAAGTGGCAAAAGCACAAAAGTCCCTGCCGCGTATGCGGCTCAAGGCTACA
AAGTGCTAGTGCTAAACCCCTCTGTTGCTGCGACTCTGGGTTTTGGGGCATATATGTCCAAGGCACATGGC
ATTGATCCCAATATACGATCAGGGGTCAGAACTATCACCACAGGCGCGCCCATCACGTACTCCACGTACGG
GAAGTTCTTGGCCGATGGAGGTTGCGCGGGGGGCGCGTATGATATCATCATCTGTGATGAATGCCATTCTA
CTGATGCAACGACGGTCCTGGGCATAGGCACGGTCTTAGACCAAGCGGAAACCGCTGGAGCGCGTCTTGTC
GTGCTCGCGACCGCTACGCCACCCGGATCGGTGACAACGCCCCACTCCAACATAGAGGAGGTCGCTTTGCC
GACGACGGGAGAGATACCTTTCTACGGCAAGGCAGTCCCCCTATCTTTGGTTAAGGGGGGCAGGCATCTCA
TCTTCTGTCACTCAAAGAAGAAGTGTGATGAGTTGGCCAAGCAACTATCATCTCTTGGCCTCAATGCGGTA
GCCTATTATAGGGGCCTTGACGTCTCAGTGATACCATTATCTGGAGACGTCGTGGTTTGCGCCACAGACGC
CCTCATGACAGGCTTCACAGGTGACTTTGACTCAGTGATAGACTGCAATACGTCTGTCATACAAACAGTTG
ACTTCAGCCTAGACCCTACTTTCACCATAGAGACCACAACCGTACCCCAGGACGCAGTGTCCCGGAGCCAA
CGGAGGGGCCGCACTGGTAGGGGGAGGTTAGGCATATACCGGTATGTCACCCCAGGAGAGAGGCCATCCGG
CATATTTGACACCTCAGTACTCTGCGAGTGCTACGATGCTGGATGCGCTTGGTATGAACTGACACCGGCAG
AGACAACGATCAGGTTAAGGGCTTATTTCAACACACCGGGCCTCCCCGTCTGCCAGGATCACCTGGAATTT
TGGGAGAGCGTCTTTACGGGTCTCACCCATATAGACGGTCATTTCCTATCCCAGACCAAACAGGCGGGTGA
CAACTACCCTTACCTGGTCGCCTACCAGGCAACAGTCTGTGCCAAGGCTTTGGCACCCCCACCCAGTTGGG
ACACAATGTGGAAATGCCTCCTCCGCCTCAAGCCAACTTTGCGGGGACCGACCCCCCTCCTTTACAGGCTG
GGGTCCGTACAAAATGAGGTGGTACTCACGCACCCGATCACCAAGTACATCATGGCCTGCATGTCTGCCGA
TCTTGAGGTTGTGACCAGCACGTGGGTCCTGGTAGGCGGTCTTCTGGCGGCCCTTGCTGCCTACTGCTTGT
CAGTGGGCAGCGTGGTAATCGTCGGGAGGGTCGTCATATCGGGCCAACCTGCTGTCATCCCCGATCGGGAG
GTGCTGTACCGACAGTTCGACGAAATGGAAGAGTGCTCTAAGCACGTTCCATTCGTCGAGCATGGCCTGCA
GCTAGCGGAGCAATTCAAACAGAAGGCCATAGGCCTTATGAGCATCGCTGGCAAGCAGGCCCAGGAAGCAG
CACCAGTGGTCCAGTCAAATTTTGCCAAACTTGAACAGTTTTGGGCGAAGCATATGTGGAACTTCATCAGT
GGTATTCAATACCTTGCCGGGCTGTCTACCTTGCCGGGCAACCCAACTATTGCTTCCCTCATGGCGTTCAC
CGCCGCGGTCACTAGCCCCCTAACGACCCAACAGACTCTCCTATTCAACATCTTGGGAGGTTGGGTGGCCT
CACAGATCGCGACCCCTACGGCCTCTACGGCTTTTGTCATAAGCGGCATTGCGGGGGCTGCGGTCGGGAGT
GTTGGCCTGGGGAAGATCCTAGTGGACATTCTTGCTGGCTACGGTGCCGGTGTGGCCGGCGCTGTGGTCAC
CTTCAAGATCATGAGCGGCGAGACACCATCAACAGAAGACTTGGTGAACTTGCTCCCAGCAATACTATCGC
CGGGAGCCCTGGTGGTAGGGGTGGTATGTGCCGCAATTTTGCGGCGTCACGTGGGACCGGGTGAGGGAGCA
GTTCAGTGGATGAACCGCTTGATCGCATTCGCGTCAAGGGGCAACCACGTGGCTCCCACACACTACGTTCC
CGAGTCCGACGCAGCGGCTCGCGTGACTGTCATACTATCATCCCTGACTGTGACCTCCCTTCTCAGACGCC
TCCACAAGTGGATCAACGAGGACTGTTCTACTCCTTGTGATCGCTCTTGGTTATGGGAGATCTGGGACTGG
GTCTGCACCGTACTGAGTGACTTTAAAACGTGGCTGAAGGCCAAGCTATTGCCTCGCATGCCCGGCATTCC
CTTCCTCTCCTGTCAGAGGGGGTACAGAGGAGTGTGGCGGGGAGATGGGGTAATGCACACAACATGCACAT
GCGGCGCAGAGCTGGCCGGCCACGTCAAAAATGGCTCGATGAGGATCGTCGGGCCCAAGACCTGCAGCAAT
ACCTGGCACGGGACCTTCCCCATCAATGCTTACACCACGGGTCCTAGCGTGCCCATCCCCGCGCCTAACTA
CAAGTTTGCGCTGTGGAGGGTATCCGCGGAGGAATACGTGGAGGTTCGCAGAGTAGGGGAGTTCCATTATA
TCACCGGGGTTACACAGGATAACATCAAGTGCCCCTGCCAGGTACCCGCACCTGAGTTCTTCACTGAGGTG
GATGGCGTCAGGCTCCATCGTCATGCCCCTGCGTGCAAGCCCATACTGAGGGACGATGTGTCCTTTACAGT
GGGCCTCAATACTTTTGTGGTGGGGTCCCAGCTCCCCTGCGAGCCCGAGCCAGACGTCGCAGTGTTAACAT
CTATGCTGACAGATCCATCTCACATCACAGCGGAGGCGGCACGCCGTAGGCTGGGAAGGGGGTCACCACCC
TCCTTGGCCAGCTCCTCGGCGAGCCAGCTATCTGCCCCATCCTTAAAAGCTACATGCACCGACCACAAAGA
CTCCCCTGGAGTGGACCTCATCGAGGCTAATCTCCTCTGGGGCGCCAATGCTACCAGGGTTGAGTCAGAGG
ATAAGGTGCTGATCTTGGACTCTTTTGAGCCCCTAGTGGCCGAGACGGATGACAGGGAGATCTCCGTCTCA
GCAGAGATCCTGCGGACTTCGAAGAAGTTCCCGAGGGCCATGCCAATTTGGGCTCAGCCAGCTTATAACCC
GCCTCTCATTGAGACGTGGAAACAACCAGACTACGAACCACCAGTCGTTCACGGCTGCGCACTGCCCCCGG
ACAAACCAACTCCTGTTCCTCCCCCCAGGAGGAAGCGGGCAGTTGCGCTCTCGGAGTCCAACATCTCAGCG
GCACTGGCGAGCTTGGCAGACAAGACCTTTAGCCAGCCAGCTGTCAGCTCCGATTCCGGAGCGGCCTTTTC
CACCCCAACTGAGACTTCTGAACCAGACCCCATCATCGTGGACGACAAATCAGACGACGGATCTTACTCGT
CAATGCCTCCGCTTGAAGGGGAGCCTGGTGACCCAGACTTGACATCAGACTCTTGGTCCACCGTCAGCGGA
TCGGAGGACGTAGTGTGCTGCTCAATGTCCTACTCGTGGACGGGGGCGCTTGTCACCCCCTGCGCAGCTGA
GGAAACCAAGCTGCCCATCAACCCCCTGAGCAACTCACTGCTACGCCATCACAACATGGTGTACTCCACGA
CTTCTCGTTCCGCCGCCACCCGGCAGAAGAAGGTCACCTTCGACCGCATGCAAGTGGTGGACAGCCATTAC
AATGAAGTACTTAAGGAGATTAAGGCACAAGCCTCCACAGTGAAGGCGCGGTTACTCACGGTTGAGGAAGC
CTGCAACCTGACGCCCCCCCACTCGGCCAGATCAAAATTTGGTTACGGGGCGAAGGAGGTTCGGAGCCATA
CCCGCAAAGCCATTAACCACATCAACTCCGTGTGGGAGGACTTGCGGGAAGACAACACTACCCCCATCCCT
ACAACAATCATGGCTAAGAATGAGGTCTTCTCCGTGACACCGGAGAAGGGCGGCAAAAAATCGGCTCGTCT
AATCGTGTACCCTGACCTAGGGGTGCGGGTGTGCGAGAAGAGGGCCCTGTATGATGCCGTCAAACAACTTT
CTCTGGCCGTGATGGGAACCTCTTACGGTTTCCAGTACTCACCATCGCAGCGGGTCGAGTTCCTTTTGAAC
GCTTGGCGTTCAAAAAAGACCCCTATGGGGTTTTCATATGACACCCGCTGCTTTGACTCCACTGTAACCGA
AAGGGACATCAGGGTTGAGGAGGAGGTCTATCAGTGTTGTGACCTAGAGCCCGAAGCCCGCAAGGTGATAT
CCGCCCTCACGGAGAGACTCTACGTGGGCGGTCCCATGTACAACAGCAGGGGAGACCTTTGCGGGATCCGA
CGGTGCCGCGCAAGCGGCGTCTTCACCACCAGCTTTGGGAACACACTAACGTGCTATCTTAAGGCCAACGC
AGCCATCAGGGCTGCAGGCCTAAAAGACTGCACCATGCTGGTTTGTGGCGACGACTTAGTCGTTATCGCTG
AAAGCGATGGCGTGGAGGAGGACAAACGTGCCCTCGGAGCCTTCACGGAGGCTATGACGAGGTACTCAGCC
CCCCCCGGAGACGCCCCACAACCAGCATATGACCTGGAGCTCATAACATCTTGCTCCTCCAATGTTTCCGT
CGCACATGATGGGACCGGCAAAAGGGTCTACTACCTGACCCGCAACCCTGAGACTCCCCTGGCACGGGCTG
CCTGGGAGACAGCTCGACACACTCCAGTCAACTCTTGGCTTGGGAACATCATAATCTACGCGCCCACCATT
TGGGTGCGCATGGTTTTGATGACCCACTTCTTCTCAATACTCCAAAGCCAGGAGGCCCTTGAGAAAGCACT
AGACTTCGACATGTACGGAGTCACATACTCTATCACTCCGCTGGACTTGCCAGCCATAATTCAAAGACTCC
ACGGCTTAAGCGCATTTACGCTGCACGGATACTCTCCACACGAACTCAACCGGGTGGCCGGAAGCCTCAGG
AAACTTGGGGTACCACCGTTGAGAGCGTGGAGACATCGGGCCCGAGCAGTCCGCGCTAAGCTCATCGCTCA
GGGGGGTAGAGCCAGAATCTGTGGCATATACCTCTTTAACTGGGCGGTAAAAACCAAAGCCAAACTCACTC
CATTGCCCGCCGCTGCCAAACTCGACCTGTCGAGTTGGTTTACGGTGGGTGCTGGCGGGGGGGACATTTAT
CACAGCGTGTCCCATGCCCGACCCCGCTACTTACTCCTGTGCCTACTCCTACTTTCCGTAGGGGTAGGCAT
CTTCCTGCTGCCCGCTCGGTAGGCAGCTTAACACTCCGACCTTAGGGTCCCCTTGTTTTTTTTTTTTTTTT
TTTTTTTTTTTTTTTTTTTTTTTTTTTTTTCCTTTCCTTCTTTCCTTTCCTAATCTTTCTTTCTTGGTGGC
TCCATCTTAGCCCTAGTCACGGCTAGCTGTGAAAGGTCCGTGAGCCGCATGACTGCAGAGAGTGCTGATAC
TGGCCTCTCTGCAGATCATGTTCTAGAGTCGACCTGCAGGCATGCAAGCTTGGCGTAATCATGGTCATAGC
TGTTTCCTGTGTGAAATTGTTATCCGCTCACAATTCCACACAACATACGAGCCGGAAGCATAAAGTGTAAA
GCCTGGGGTGCCTAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGGACAGTATTTGGTATCT
GCGCTCTGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCATCGTGGTGTCACGCT
CGTCGTTTGGTATGGCTTCATTCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAAGTTGGCCGCAGTGTTATCACT
CATGGTTATGGCAGCACTGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGTTATTG
TCTCATGAGCGGATACATATTTGAATGTATTTAGAAAAATAAACAAATAGGGGTTCCGCGCACATTTCCCC
GAAAAGTGCCACCTGACGTCTAAGAAACCATTATTATCATGACATTAACCTATAAAAATAGGCGTATCACG
AGGCCCTTTCGTCTCGCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAGCGGGTGTTGGCGGGT
GTCGGGGCTGGCTTAACTATGCGGCATCAGAGCAGATTGTACTGAGAGTGCACCATATGCGGTGTGAAATA
CCGCACAGATGCGTAAGGAGAAAATACCGCATCAGGCGCCATTCGCCATTCAGGCTGCGCAACTGTTGGGA
AGGGCGATCGGTGCGGGCCTCTTCGCTATTACGCCAGCTGGCGAAAGGGGGATGTGCTGCAAGGCGATTAA
GTTGGGTAACGCCAGGGTTTTCCCAGTCACGACGTTGTAAAACGACGGCCAGTGAATTCTAATACGACTCA
CTATA

SEQ ID NO: 2 provides the polyprotein sequence for GT4d isolate QC382 (accession ACS29436). The following table further annotates the starting and ending positions of each individual protein.

```
Protein 1 . . . 3006
Regions - Proteins
      2 . . . 115 - HCV_capsid (Hepatitis C virus capsid protein)
  116 . . . 190 - HCV_core (Hepatitis C virus core protein)
  195 . . . 382 - HCV_env (Hepatitis C virus envelope glycoprotein)
  387 . . . 728 - HCV_NS1 (Hepatitis C virus non-structural protein E2/NS1)
  810 . . . 1004 - HCV_NS2 (Hepatitis C virus non-structural protein NS2)
1056 . . . 1203 - Peptidase_S29 (Hepatitis C virus NS3 protease)
1223 . . . 1350 - DEXDc (DEAD-like helicases superfamily)
1227 . . . 1354 - DEXDc (DEAD-like helicases superfamily)
1377 . . . 1462 - HELICc(Helicase superfamily c-terminal domain)
1657 . . . 1710 - HCV_NS4a (Hepatitis C virus non-structural protein NS4a)
1727 . . . 1920 - HCV_NS4b (Hepatitis C virus non-structural protein NS4b)
1974 . . . 1995 - HCV_NS5a (Hepatitis C virus non-structural 5a protein
membrane anchor)
2005 . . . 2066 - HCV_NS5a_1a (Hepatitis C virus non-structural 5a zinc
finger domain)
2067 . . . 2167 - HCV_NS5a_1b (Hepatitis C virus non-structural 5a domain
1b)
2178 . . . 2415 - HCV_NS5a_C (HCV NS5a protein C-terminal region)
2418 . . . 2929 - RdRP_3 - (Viral RNA dependent RNA polymerase)
2532 . . . 2813 - RNA_dep_RNAP (RNA_dep_RNAP: RNA-dependent RNA
polymerase)
SEQ ID NO: 2
MSTNPKPQRKTKRNTNRRPMDVKFPGGGQIVGGVYLLPRRGPRLGVRATRKTSERSQPRGRRQPIPKARQ
PEGRSWAQPGYPWPLYGNEGCGWAGWLLSPRGSRPSWGPNDPRRRSRNLGKVIDTLTCGFADLMGYIPVV
GAPVGGVARALAHGVRLLEDGVNYATGNLPGCSFSIFLLALLSCLTVPASAYNYRNSSGVYHVTNDCPNS
SIVYEADHHILHLPGCVPCVRVGNKSTCWVSLTPTVAAPYLNAPLESLRRHVDLMVGAATLCSALYIGDV
CGGAFLVGQLFTFQPRRHWTTQDCNCSIYTGHITGHRMAWDMMMNWSPTTTLVLAQLMRIPSAMVDLLAG
GHWGILVGIAYFSMQANWATVILVLFLFAGVDAETIVSGGQAGRTMFGFTSLLNLGPSQKLQLINTNGSW
HINRTALNCNDSLNTGLIASLFYAHRFNSSGCPERLASCRSLDSFQQGWGPLGIYQANQSDTRPYCWNYT
PQPCWTVPASTVCGPVYCFTPSPVVVGTTDRLGVPTYTWGENETDVFLLNSTRPPRGAWFGCTWMNGTGF
TKSCGGPPCRITTINNTWGCPTDCFRKHPEATYIKCGSGPWLTPRCLVHYPYRLWHYPCTVNYTIFKIRM
YVGGIEHRLDVACNWTRGEPCDLEHRDRAEISPLLLSTTQWQVLPCSFTTLPALSTGLIHLHQNIVDVQY
LYGVGSAVVSWALKWEYIVLAFLLLADARLCACLWMMLMVSQVEAALANLITINAVSVAGIHGFWYAIFV
ICIAWHVKGRFPAAVTYAACGLWPLLLLVLMLPERAYAFDREIAGSAGGGVLVLLTLLTLSSHYKQWLAR
GIWWLQYFIARAEAITHVYVPSLDVRGPRDSIIILTALAFPHVAFETTKHLLAILGPLYILQASLLCVPY
FVRAHALVKLCSLVRGVMCGKYCQMALLKIGALTGTYVYNHLTPLSDWAAEGLNDLAVALEPVVFTAMEK
KIITWGADTAACGDILQGLPVSARLGNEILLGPADAHATRGWRLLAPITAYAQQTRGTLGTIITSLTGRD
TNENCGEIQVLSTATQSFLGSAINGVMWTVYHGAGSKTISGPKGPVNQMYTNVDQDLVGWPAPPGVKSLA
PCTCGSSDLFLVTRHADVVPVRRRGDTRGALLSPRPISTLKGSSGGPLLCPLGHVAGIFRAAVCTRGVAK
AVDFVPVESLETTMRSPVFSDNSTPPAVPQTYQVAHLHAPTGSGKSTKVPAAYAAQGYKVLVLNPSVAAT
LGFGAYMSKAHGIDPNIRSGVRTITTGAPITYSTYGKFLADGGCAGGAYDIIICDECHSTDATTVLGIGT
VLDQAETAGARLVVLATATPPGSVTTPHSNIEEVALPTTGEIPFYGKAVPLSLVKGGRHLIFCHSKKKCD
ELAKQLSSLGLNAVAYYRGLDVSVIPLSGDVVVCATDALMTGFTGDFDSVIDCNTSVIQTVDFSLDPTFT
IETTTVPQDAVSRSQRRGRTGRGRLGIYRYVTPGERPSGIFDTSVLCECYDAGCAWYELTPAETTIRLRA
YFNTPGLPVCQDHLEFWESVFTGLTHIDGHFLSQTKQAGDNYPYLVAYQATVCAKALAPPPSWDTMWKCL
LRLKPTLRGPTPLLYRLGSVQNEVVLTHPITKYIMACMSADLEVVTSTWVLVGGLLAALAAYCLSVGSVV
IVGRVVISGQPAVIPDREVLYRQFDEMEECSKHVPFVEHGLQLAEQFKQKAIGLMSIAGKQAQEAAPVVQ
SNFAKLEQFWAKHMWNFISGIQYLAGLSTLPGNPTIASLMAFTAAVTSPLTTQQTLLFNILGGWVASQIA
TPTASTAFVISGIAGAAVGSVGLGKILVDILAGYGAGVAGAVVTFKIMSGETPSTEDLVNLLPAILSPGA
LVVGVVCAAILRRHVGPGEGAVQWMNRLIAFASRGNHVAPTHYVPESDAAARVTVILSSLTVTSLLRRLH
KWINEDCSTPCDRSWLWEIWDWVCTVLSDFKTWLKAKLLPRMPGIPFLSCQRGYRGVWRGDGVMHTTCTC
GAELAGHVKNGSMRIVGPKTCSNTWHGTFPINAYTTGPSVPIPAPNYKFALWRVSAEEYVEVRRVGEFHY
ITGVTQDNIKCPCQVPAPEFFTEVDGVRLHRHAPACKPILRDDVSFTVGLNTFVVGSQLPCEPEPDVAVL
TSMLTDPSHITAEAARRRLGRGSPPSLASSSASQLSAPSLKATCTDHKDSPGVDLIEANLLWGANATRVE
SEDKVLILDSFEPLVAETDDREISVSAEILRTSKKFPRAMPIWAQPAYNPPLIEXWKQPDYEPPVVHGCA
LPPDKPTPVPPPRRKRAVALSESNISAALASLADKTFXQPAVSSDSGAAFSTPTETSEPDPIIVDDKSDD
GSYSSMPPLEGEPGDPDLTSDSWSTVSGSEDVVCCSMSYSWTGALVTPCAAEETKLPINPLSNSLLRHHN
MVYSTTSRSAATRQKKVTFDRMQVVDSHYNXVLKEIKAQASTVKARLLTVEEACNLTPPHSARSKFGYGA
KEVRSHTRKAINHINSVWEDLREDNTTPIPTTIMAKNEVFSVTPEKGGKKSARLIVYPDLGVRVCEKRAL
YDAVKQLSLAVMGTSYGFQYSPSQRVEFLLNAWRSKKTPMGFSYDTRCFDSTVTERDIRVEEEVYQCCDL
EPEARKVISALTERLYVGGPMYNSRGDLCGIRRCRASGVFTTSFGNTLTCYLKANAAIRAAGLKDCTMLV
CGDDLVVIAESDGVEEDKRALGAFTEAMTRYSAPPGDAPQPAYDLELITSCSSNVSVAHDGTGKRVYYLT
RNPETPLARAAWETARHTPVNSWLGNIIIYAPTIWVRMVLMTHFFSILQSQEALEKALDFDMYGVTYSIT
PLDLPAIIQRLHGLSAFTLHGYSPHELNRVAGSLRKLGVPPLRAWRHRARAVRAKLIAQGGRARICGIYL
FNWAVKTKAKLTPLPAAAKLDLSSWFTVGAGGGDIYHSVSHARPRYLLLCLLLLSVGVGIFLLPAR
```

Thus, in one aspect, a genotype 4d HCV RNA construct is provided, comprising a 5′NTR, an internal ribosome entry site (IRES), sequences encoding NS3, NS4A, NS4B, NS5A and NS5B, and a 3′NTR, wherein the construct is capable to replicate in a eukaryotic cell. In one aspect, the construct comprises an adaptive mutation in NS3, NS4A, NS4B, NS5A or NS5B.

In any of the above embodiments, the HCV RNA can further comprise a marker gene for selection. A non-limiting example of such marker gene is a neomycin phosphotransferase gene. Other examples are well known in the art.

In any of the above embodiments, the HCV RNA can further comprise a reporter gene. A non-limiting example of such marker gene is a luciferase gene. Other examples are well known in the art.

The RNA construct of any of the above embodiment can further comprise sequences encoding one or more of C, E1 or E2. In one aspect, the RNA construct is a full-length HCV replicon.

The disclosure also provides a single or double-stranded DNA that can be transcribed to a RNA construct of any of the above embodiment, a viral particle comprising a RNA construct of any of the above embodiment, or an isolated cell comprising a RNA construct of any of the above embodiment.

Also provided are mutant proteins as identified herein and their homologues. In one embodiment, provided is an NS4A protein of HCV genotype 4d that comprises an arginine at residue 34. In one aspect, the disclosure provides a protein that has at least 90% sequence, or at least 95%, identity to 1657-1710 of SEQ ID NO: 2 and has an arginine at residue 34 relative to NS4A.

In one embodiment, provided is an NS5A protein of HCV genotype 4d that comprises a glycine or isoleucine at residue 232. In one aspect, the disclosure provides a protein that has at least 90% sequence, or at least 95%, identity to 1974-1995 of SEQ ID NO: 2 and has a glycine or isoleucine at residue 232 relative to NS5A.

In yet another aspect, provided is a polynucleotide encoding the protein of any of such embodiments. The polynucleotide can be RNA or DNA. In another aspect, provided is an RNA or DNA construct comprising the polynucleotide. In yet another aspect, provided is a cell comprising the polynucleotide. Still in one aspect, provided is an antibody that specifically recognizes a protein of any of the above embodiments.

### HCV Genotype 4d Replicons and Cells Containing the Replicons

Another embodiment of the present disclosure provides an isolated cell comprising a genotype 4d hepatitis C viral (HCV) RNA that replicates in the cell. In one aspect, there is an absence, in the cell, of a DNA construct encoding the RNA and thus copies of the HCV RNA are not transcribed from a DNA, such as cDNA, construct.

In one aspect, the cell comprises at least 10 copies of the RNA. In another aspect, the cell comprises at least 100, 500, 1000, 2000, 5000, 10,000, 1×10⁵, 1×10⁶, 1×10⁷, 1×10⁸ or 1×10⁹ copies of the RNA.

The HCV RNA can be subgenomic HCV sequence or a full-length HCV sequence. In either case, RNA can include one or more of 5′NTR, an internal ribosome entry site (IRES), sequences encoding NS3, NS4A, NS4B, NS5A and NS5B, and a 3′NTR.

In any of the above embodiments, the HCV RNA can include an adaptive mutation that enables the RNA to replicate in the cell. Such adaptive mutations can include NS3 (E176G, A240V), NS4A (Q34R) and/or NS5A (S232G/I). In some embodiments, the mutations include either or both of NS4A (Q34R) and/or NS5A (S232G/I). In some embodiments, the mutations do not include NS3 (E176G and A240V).

Also contemplated are that the HCV RNA can be a RNA sequence that has at least about 75%, or about 80%, 85%, 90%, 95%, 98%, 99%, or about 99.5% sequence identity to any of the disclosed sequences, so long as it retains the corresponding adaptive mutation(s).

In one aspect, the cell is a eukaryotic cell such as a mammalian cell and in particular a human cell. In another aspect, the cell is hepatoma cell, such as but not limited to a Huh7 cell (e.g., Huh7-Lunet, 51C and 1C). It is herein discovered surprisingly that Huh7 1C cell is particularly permissive to the genotype 4d replicons and thus in one aspect, the cell is a Huh7 1C cell. In some aspects, the cell is placed at an in vitro or ex vivo condition.

### Methods of Preparing Genotype 4d Replicons

After HCV genotype 4d replicons are identified, as shown in Example 1, introduction of the relevant adaptive mutation into a corresponding genotype HCV RNA can result in the RNA's capability to replicate, in particular in a mammalian cell in vitro. Accordingly, the present disclosure provides a method of improving the capability of a genotype 4d HCV viral RNA to replicate in a eukaryotic cell, comprising one or more of: (a) substituting residue 34 of NS4A with an arginine, (b) substituting residue 176 of NS3 with glycine, (c) substituting residue 240 of NS3 with valine, and/or (d) substituting 232 of NS5A with glycine or isoleucine. In one aspect, the method comprises at least two substitutions of (a)-(d). In one aspect, the method entails (a) substituting residue 34 of NS4A with an arginine, and/or (b) substituting residue 240 of NS3 with valine, but keeping the E176 and A240 residues of NS3 wild-type, i.e., not mutating these amino acid residues.

### Methods of Screening HCV Inhibitors Targeting Genotype 4d

Numerous known and unknown HCV inhibitors have been tested for their efficiency in inhibiting the genotype 4d HCV, in comparison with genotype 1b (Example 1). Some showed higher efficacy for genotype 4d, and some were not as efficacious. The usefulness of the new identified genotype 4d replicons, therefore, is adequately demonstrated.

Thus, the present disclosure also provides, in one embodiment, a method of identifying an agent that inhibits the replication or activity of a genotype 4d HCV, comprising contacting a cell of any embodiment of the present disclosure with a candidate agent, wherein a decrease of replication or a decrease of activity of a protein encoded by the RNA indicates that the agent inhibits the replication or activity of the HCV. In some aspects, the protein is one or more of NS3, NS4A, NS4B, NS5A or NS5B. Replication of the RNA, in one aspect, can be measured by a reporter gene on the RNA, such as the luciferase gene.

Provided in another embodiment is a method of identifying an agent that the activity of a genotype 4d HCV, comprising contacting the lysate of a cell of any embodiment of the present disclosure with a candidate agent, wherein a decrease of the activity of a protein encoded by the RNA indicates that the agent inhibits the activity of the HCV. In one aspect, the protein is one or more of NS3, NS4A, NS4B, NS5A or NS5B. In another aspect, the method further comprises measuring the replication of the RNA or the activity of the protein encoded by the RNA.

A HCV inhibitor (or “candidate agent”) can be a small molecule drug that is an organic compound, a peptide or a protein such as antibodies, or nucleic acid-based such as siRNA. In May 2011, the Food and Drug Administration approved 2 drugs for Hepatitis C, boceprevir and telaprevir. Both drugs block an enzyme that helps the virus reproduce. Boceprevir is a protease inhibitor that binds to the HCV NS3 active site on hepatitis C genotype 1. Telaprevir inhibits the hepatitis C virus NS3/4A serine protease.

More conventional HCV treatment includes a combination of pegylated interferon-alpha-2a or pegylated interferon-alpha-2b (brand names Pegasys or PEG-Intron) and the antiviral drug ribavirin. Pegylated interferon-alpha-2a plus ribavirin may increase sustained virological response among patients with chronic hepatitis C as compared to pegylated interferon-alpha-2b plus ribavirin according to a systematic review of randomized controlled trials.

All of these HCV inhibitors, as well as any other candidate agents, can be tested with the disclosed methods for their efficacy in inhibiting HCV genotype 4d. The cells are then incubated at a suitable temperature for a period time to allow the replicons to replicate in the cells. The replicons can include a reporter gene such as luciferase and in such a case, at the end of the incubation period, the cells are assayed for luciferase activity as markers for replicon levels. Luciferase expression can be quantified using a commercial luciferase assay.

Alternately, efficacy of the HCV inhibitor can be measured by the expression or activity of the proteins encoded by the replicons. One example of such proteins is the NS3 protease, and detection of the protein expression or activity can be carried out with methods known in the art, e.g., Cheng et al., Antimicrob Agents Chemother 55:2197-205 (2011).

Luciferase or NS3 protease activity level is then converted into percentages relative to the levels in the controls which can be untreated or treated with an agent having known activity in inhibiting the HCV. A decrease in HCV replication or decrease in NS3 activity, as compared to an untreated control, indicates that the candidate agent is capable of inhibiting the corresponding genotype of the HCV. Likewise, a larger decrease in HCV replication or larger decrease in NS3 activity, as compared to a control agent, indicates that the candidate is more efficacious than the control agent.

### EXAMPLES

The present disclosure is further defined by reference to the following examples. It will be apparent to those skilled in the art that many modifications, both to threads and methods, may be practiced without departing from the scope of the current disclosure.

### ABBREVIATIONS

Unless otherwise stated all temperatures are in degrees Celsius (° C.). Also, in these examples and elsewhere, abbreviations have the following meanings:

| | | |
|---|---|---|
| μF | = | MicroFaraday |
| μg | = | Microgram |
| μL | = | Microliter |
| μM | = | Micromolar |
| g | = | Gram |
| hr | = | Hour |
| mg | = | Milligram |
| mL | = | Milliliter |
| mM | = | Millimolar |
| mmol | = | Millimole |
| nM | = | Nanomolar |
| nm | = | Nanometer |
| pg | = | pictograms |
| DMEM | = | Dulbecco's modified Eagle's medium |
| EMCV | = | encephalomyocarditis virus |
| FBS | = | fetal bovine serum |
| HCV | = | Hepatitis C virus |
| IRES | = | internal ribosome entry site |
| rpm | = | revolutions per minute |
| RT-PRC | = | reverse transcription-polymerase chain reaction |

### Example 1

### Generation of Robust Genotype 4d Hepatitis C Virus Subgenomic Replicons

This example shows that adaptive mutations were identified from genotype 4d HCV viral replicons capable of replication in cells and that HCV replicons with these adaptive mutations are useful tools for antiviral drug screening.

FIG. 1A-B illustrate the process of generation of GT 4d-Neo subgenomic replicon colonies in different types of cell lines, Huh7-Lunet, 1C, 4a-Cure and 3a-Cure. The 1C cells turned out to be the most permissive, the colonies from which were obtained and the RNA concentration confirmed with RT-PCR.

Three colonies, 4d-1C-1, 4d-1C-2 and 4d-1C-3, were further analyzed. RNA was extracted from these colonies (FIG. 2A) and was retransfected. The transfected colonies were then examined with respect to NS3 activity and NS5A staining and the RNAs were sequenced (FIG. 2B).

Two candidate HCV inhibitors, Compound A (FIG. 3A) and B (FIG. 3B) were used to test the inhibition of NS3 activities of the replicons isolated from pooled colonies (4d-2Re and 4d-3Re, see FIG. 2). 4d-3Re and 4d-2Re showed dose dependent inhibition of NS3 activity by Compound A (FIG. 3A), and a slight inhibition at high concentration of Compound B (FIG. 3B). Also observed was that NS3 activity was higher in 4d-3Re than 4d-2Re.

RNA's extracted from the individual colonies and pooled one were sequenced to identify adaptable mutation. The following table shows the identified mutations.

| | Mutations | | | | |
|---|---|---|---|---|---|
| Samples | NS3 | NS4A | NS4B | NS5A | NS5B |
| 4d-1C-2 | T591I | Q34Q/R | S258S/P | K247E | E87D |
| (1st transfection) | | | | | |
| 4d-1C-3 | D81N/D, | Q34R | | S232G | |
| (1st transfection) | R119K/R | | | (S2204G) | |
| 4d-3Re | | Q34Q/R | | S232G | |
| (re-transfection) | | | | (S2204G) | |

Sequences from NS3 to NS5B of the GT 4d colonies matched with the 4d plasmid sequence well. Q34R was identified in both 4d-1C-2 and 4d-1C-3 colonies. S232G was identified in colony 4d-1C-3, which demonstrated higher NS3 activity than 4d-1C-2.

In this example, therefore, GT 4d-Neo stable subgenomic replicons were established. Adaptive mutations Q34R and S232G were identified in GT 4d replicons. Further, high levels of NS3 activity and NS5A expression were observed and dose dependent inhibition of Compound A (a known HCV inhibitor, Sofosbuvir) was observed in these GT 4d replicons.

FIG. 4 shows the comparison results of replication levels among GT-4d-Neo colonies, measured with NS3 activity. 4000 cells/well were plated in 96-well white plates. NS3 activity was read 72 hours after plating. Values shown in FIG. 4 are mean of DMSO treated well from 3 plates. 4d-3 showed the highest NS3 activity over all, which harbored the Q34R and S232G adaptive mutations.

Constructs were prepared with Pi-Rluc and Rluc-Neo reporter genes. FIG. 5A shows such a design. Mutations incorporated into the constructs are shown in the table below. Wild-type of 4d NS3 has an AscI site. A silent mutation was introduced to knock it out (FIG. 5B). FIG. 5C illustrates the detailed replacement process of Neo with Rluc-Neo/Pi-Rluc.

A total of 11 Pi-Rluc and 3 Rluc-Neo in-Fusion were performed (FIG. 5D). Miniprep of 2 colonies were prepared from each transformation for Pi-Rluc/Rluc-Neo and subject to NS3 to NS5B sequencing.

The replication time course of the replicons were measured. FIG. 6 shows the generation of replication time course for adaptive mutations in GT4d Pi-Rluc replicon. Shown in FIG. 7 are the replication curves of 4d Pi-Rluc replicons carrying single adaptive mutations. Compared to 1b Pi-Rluc (positive control), none of the 4d wild-type or with single mutations showed good replication time course.

By contrast, replication of 4d Pi-Rluc replicons carrying double adaptive mutations (Q34R+S232I or Q34R+S232G) was greatly higher (FIG. 8). Further, the replication curves of 4d Pi-Rluc replicons carrying double, triple and all 4 adaptive mutations are shown in FIG. 9. As shown in the figure, the replicons with triple and quadruple mutation did not replicate as efficiently as those with double mutations.

FIG. 10 compares the replication capacity of different replicons at 96 hours post transfection. Apparently, replicons with the two double mutations showed the highest replication capability. Similar comparison is shown in FIG. 11, for replicons at 120 hours post transfection.

Stable GT4d subgenomic replicons were prepared to include these double mutations (FIG. 12). Ten micrograms of in vitro transcribed 4d Rluc-Neo RNA were transfected into 1C cells. G418 selection started 2 days after transfection and plates were fixed and stained after 2 weeks of G418 selection. As shown in the figure, both replicons exhibited high replication capacity, with Q34R+S232G being even better. The luciferase activity of these stable replicon cells of these replicons were further compared to GT4a replicons and GT1b. As shown in FIG. 14, their replication capacities were comparable.

Another comparison was made, with respect to each replicon's susceptibility against HCV antiviral agents. The results are shown in the table below.

| | | EC₅₀ (nM) n = 2 | | | |
|---|---|---|---|---|---|
| | | | | GT4d | GT4d |
| | | | | (34R + | (34R + |
| Inhibitor | | | | 232G- | 232I- |
| Class | Compound | GT1b | GT4a | pool) | pool) |
| NS3 | C | 9.2 | 32 | 19 | 38 |
| protease | D | 425 | 1971 | 1080 | 1424 |
| | E | 481 | 2849 | 2110 | 2500 |
| | F | 0.39 | 1.16 | 0.84 | 1.38 |
| NS5A | G | 0.004 | 0.006 | 0.012 | 0.007 |
| | H | 0.002 | 0.29 | 0.60 | 0.57 |
| | I | 0.008 | 0.015 | 0.22 | 0.22 |
| NS5B Nuc | A | 158 | 70 | 33 | 37 |
| | J | 12297 | 11976 | 3637 | 9673 |
| NSSB | B | 1.31 | 492 | 1856 | 1569 |
| Non | K | 56 | 2457 | 1642 | >10000 |
| Nuc | | | | | |
| RBV | L | 18188 | 5148 | 2259 | 4771 |

It will be appreciated that those skilled in the art will be able to devise various arrangements which, although not explicitly described or shown herein, embody the principles of the disclosure and are included within its spirit and scope. Furthermore, all conditional language recited herein is principally intended to aid the reader in understanding the principles of the disclosure and the concepts contributed by the inventors to furthering the art, and are to be construed as being without limitation to such specifically recited conditions. Moreover, all statements herein reciting principles, aspects, and embodiments of the disclosure are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1. An isolated genotype 4d hepatitis C viral (HCV) RNA construct comprising a 5′NTR, an internal ribosome entry site (IRES), sequences encoding one or more of NS3, NS4A, NS4B, NS5A or NS5B, and a 3′NTR, wherein the RNA construct further comprises a mutation, as compared to a wild-type HCV 4d sequence, selected from Q34R in NS4A or S232G or S232I in NS5A, or combinations thereof.

2. The RNA construct of claim 1, wherein the mutation is Q34R in NS4A.

3. The RNA construct of claim 1, wherein the mutation is S232G or S232I in NS5A.

4. The RNA construct of claim 1, wherein the mutation is Q34R in NS4A and S232G or S232I in NS5A.

5. The RNA construct of claim 1, wherein the mutation is Q34R in NS4A and S232G in NS5A.

6. The RNA construct of claim 4, wherein the construct comprises wild-type amino acids at residue E176 or A240 in NS3, or both.

7. The RNA construct of claim 1, further comprising a marker gene for selection.

8. The RNA construct of claim 7, wherein the marker gene is a neomycin phosphotransferase gene.

9. The RNA construct of claim 1, further comprising a reporter gene.

10. The RNA construct of claim 9, wherein the reporter gene is luciferase.

11. The RNA construct of claim 1, wherein the construct comprises, from 5′ to 3′, the 5′NTR, the IRES, sequences encoding NS3, NS4A, NS4B, NS5A and NS5B, and the 3′NTR.

12. The RNA construct of claim 1, further comprising a sequence encoding one or more of C, E1 or E2.

13. A genotype 4d hepatitis C viral (HCV) RNA construct comprising a nuclei acid sequence of SEQ ID NO: 1 or a polynucleotide having at least 95% sequence identity to SEQ ID NO: 1, wherein the construct comprises an arginine at residue 34 in NS4A and a glycine or isoleucine at residue 232 in NS5A.

14. The RNA construct of claim 13, wherein the polynucleotide comprises a glycine at residue 232 in NS5A.

15. The RNA construct of claim 13, wherein the construct comprises wild-type amino acids at residue E176 or A240 in NS3, or both.

16. The RNA construct of claim 1, wherein the RNA construct is capable of replication in vitro.

17-28. (canceled)

29. An isolated cell comprising a genotype 4d hepatitis C viral (HCV) RNA that replicates in the cell.

30. The cell of claim 29, wherein there is an absence, in the cell, of a DNA construct encoding the RNA.

31. The cell of claim 29, wherein the cell comprises at least 10 copies of the RNA.

32. The cell of claim 29, wherein the RNA comprises a subgenomic HCV sequence.

33. The cell of claim 30, wherein the RNA comprises a 5′NTR, an internal ribosome entry site (IRES), sequences encoding NS3, NS4A, NS4B, NS5A and NS5B, and a 3′NTR.

34. The cell of claim 29, wherein the RNA comprises a full genome HCV sequence.

35. The cell of claim 29, wherein the cell is a mammalian cell.

36. The cell of claim 35, wherein the cell is a hepatoma cell.

37. The cell of claim 35, wherein the cell is a Huh7 1C cell.

38-44. (canceled)
